(19) United States
(12) Patent Application Publication
(10) Pub. No.: US 2001/0036955 A1
(43) Pub. Date: Nov. 1, 2001

(54) Method of inhibiting angiogenesis

(75) Inventors: Mary E. Gerritsen, San Mateo, CA (US); Xiaohua E. Xin, San Francisco, CA (US)

Correspondence Address: BRINKS HOFER GILSON & LIONE, P.O. BOX 10395, CHICAGO, IL, 60610, US

(73) Assignee: Genentech, Inc.

(21) Appl. No.: 09/865,859

(22) Filed: May 25, 2001

Related U.S. Application Data
(63) Continuation of application No. 09/443,010, filed on Nov. 17, 1999, now abandoned.
(60) Provisional application No. 60/116,530, filed on Jan. 20, 1999.
(60) Provisional application No. 60/109,328, filed on Nov. 20, 1998.

(51) Int. Cl. A61K 31/426

(52) U.S. Cl. 514/369

(57) ABSTRACT

Angiogenesis is inhibited and the growth of tumors is treated by administering an effective amount of a PPAR gamma ligand/agonist, optionally with an RXR receptor ligand.

### RELATED APPLICATIONS

This application is a continuation of and claims priority to Ser. No. 09/443,010, filed Nov. 17, 1999, which claimed priority to Ser. No. 60/116,530, filed Jan. 20, 1999, and Ser. No. 60/109,328, filed Nov. 20, 1998. The entire disclosure of each is hereby incorporated by reference.

### BACKGROUND OF THE INVENTION

1. Field of the Invention

The present invention relates to peroxisome proliferator activated receptor (PPAR) gamma ligands and to methods of using the ligands for diagnostic and therapeutic purposes.

2. Discussion of the Background

The two major cellular components of the vasculature are the endothelial and smooth muscle cells. The endothelial cells form the lining of the inner surface of all blood vessels, and constitute a nonthrombogenic interface between blood and tissue. In addition, endothelial cells are an important component for the development of new capillaries and blood vessels. Thus, endothelial cells proliferate during the angiogenesis, or neovascularization, associated with tumor growth and metastasis, and a variety of non-neoplastic diseases or disorders.

In the case of tumor growth, angiogenesis appears to be crucial for the transition from hyperplasia to neoplasia, and for providing nourishment to the growing solid tumor. Folkman, et al., Nature 339:58 (1989). The complex steps involved in new blood vessel formation have been well characterized in recent years. They involve degradation of the basement membrane by cellular proteases, penetration and migration of endothelial cells into the extracellular matrix, endothelial proliferation, and the formation of interconnected vascular networks. Angiogenesis also allows tumors to be in contact with the vascular bed of the host, which provides a route for metastasis of the tumor cells. It is now established that the progression of solid tumor growth and metastasis depend on angiogenesis. Evidence for the role of angiogenesis in tumor metastasis is provided, for example, by studies showing a correlation between the number and density of microvessels in histologic sections of invasive human breast carcinoma and actual presence of distant metastases. Weidner, et al., (1991) New Engl. J. Med., 324:1. This realization has prompted a broad search for new angiostatic therapies to treat cancer.

Various naturally occurring polypeptides reportedly induce the proliferation of endothelial cells. Among those polypeptides are the basic and acidic fibroblast growth factors (FGF), Burgess and Maciag, (1989) Annual Rev. Biochem., 58:575; platelet-derived endothelial cell growth factor (PD-ECGF), Ishikawa, et al., (1989) Nature, 338:557; and vascular endothelial growth factor (VEGF), Leung, et al., (1989) Science 246:1306; Ferrara& Henzel, (1989) Biochem. Biophys. Res. Commun. 161:851; Tischer, et al., (1989) Biochem. Biophys. Res. Commun. 165:1198; Ferrara, et al., PCT Pat. Pub. No. WO 90/13649 (published Nov. 15, 1990).

VEGF not only stimulates vascular endothelial cell proliferation, but also induces vascular permeability and angiogenesis. Angiogenesis is an important component of a variety of diseases and disorders including tumor growth and metastasis, rheumatoid arthritis, psoriasis, atherosclerosis, diabetic retinopathy, retrolental fibroplasia, neovascular glaucoma, age-related macular degeneration, hemangiomas, immune rejection of transplanted corneal tissue and other tissues, and chronic inflammation.

Peroxisome proliferators are agents that induce peroxisomal proliferation. Peroxisome proliferator-activated receptors (PPARs) are members of the steroid receptor superfamily. They are ligand-activated transcription factors. Three mammalian subtypes of PPAR, alpha, beta (also known as delta), and gamma have been identified and cloned. Two isoforms of PPAR gamma exist, PPAR gamma 1 and gamma 2 (Vidal-Puig et al., (1997) J. Biol. Chem:, 272:8071-8076). These two proteins differ only in their amino-terminal-30 amino acids and are the result of alternative promoter usage and differential mRNA splicing. All PPARs mediate transcriptional regulation using a central DNA binding domain which recognizes elements in the promoters of specific genes. Activation of PPAR gamma has been linked to adipocyte differentiation (Spiegelman, B. (1998) Diabetes 47:507-514; Forman, B. et al., (1995) Cell 83:803-812) and regulation of glucose homeostasis in rodents and humans. The compounds 15d-PGJ₂ (Forman, B. et al., (1995) Cell 83:803-812; Khiewer, S. et al., (1995) Cell 83:813-819) and troglitazone (Spiegelman, B. (1998) Diabetes 47:507-514), which are specific ligands for PPAR gamma, are known to inhibit macrophage activation (Ricota, M. et al., (1998) Nature (London) 391:79-82), moncyte cytokine production (Jiang, C. et al., (1998) Nature (London) 391:82086), activation of aortic smooth muscle cells (Staels, B. et al., (1998) Nature (London) 393:790-793), and the growth of human cancer cells (Elstner, E. et al., (1998) Proc. Natl. Acad. Sci. USA 95:8806-8811; Kubota, T. et al., (1998) 58:3344-3352). Whereas the PPAR gamma seems to be important in lipid storage in adipose tissue, PPAR alpha activation results in lipid catabolism (Issemann, I., and Green, S. (1990) Nature (London) 347:645-50.). Several selective PPAR alpha activators have been described, including WY 14643, clofibrate, and 8-(S)-hydroxyeicosatetraenoic acid (8(S)-HETE) (Forman, B. et al., (1997) Proc. Natl. Acad. Sci. USA 94:43124317). Specific functions for PPAR beta are not known at the present time.

It is known that the PPAR can heterodimerize with at least one other member of the steroid receptor superfamily, namely the retinoic acid receptors (RXR) (Spiegelman, B. (1998) Diabetes 47:507-514). Specific ligands for the PPAR and RXR receptors have been shown to act synergistically to inhibit cancer cell growth (Elstner, E. et al., (1998) Proc. Natl. Acad. Sci. USA 95:8806-8811) and adipocyte differentiation (Spiegelrnan, B. (1998) ibid) in vitro and to enhance insulin sensitivity in diabetic animals (Mukherjee, R. et al., (1997) Nature (London 386:407-410).

Angiogenesis, the formation of new blood vessels from pre-existing vasculature, plays important role in a variety of normal and pathological conditions including organ development, wound healing and tumor growth and metastasis. One of the important steps involved in the regulation of angiogenesis is the local degradation of the basal lamina of the pre-existing vessel wall, followed by endothelial cell invasion into the surrounding stroma (Stetler-Stevenson, W. G., 1999, J. Clin. Invest. 103:1237-1241.). This process is controlled by the expression and activation of a variety of proteolytic enzymes, including the matrix metalloproteinases (MMP), which facilitate degradation and/or invasion of the basement membrane.

MMPs, a family of more than 17 different zinc-requiring proteinases, are classified according to substrate specificity and primary structure (Westermarck, J., and Kahari V. 1999, FASEB J. 13:781-792). Endothelial cells express constitutively or can secrete after stimulation four major MMP isoforms: interstitial collagenase (MMP-1), gelatinase A (72 kDa type IV collagenase, MMP-2) and gelatinase B (92 kDa type IV collagenase, MMP-9), stromelysin-1 (MMP-3), and membrane-type MMPs (MT-MMPs) (Hanemaijer, R., Koolwijk, P., Clercq, L. L., De Vree, W. J. A., and Van Hinsbergh, V. W. M. 1993, Biochem. J. 296:803-809; Hass, T. L., bavis, S. J., and Madri, J. A. 1998 J. Biol. Chem. 273:3604-3610). The activities of these enzymes are regulated by multiple mechanisms including gene transcription, proenzyme activation and inhibition by specific endogenous TIMPs (Moses, M. A. 1997, Stem Cells 15: 180-189). The expression and secretion of MMP-1, -2, -3 and -9 by endothelial cells can be modulated by growth factors and cytokines (e.g. tumor necrosis factor alpha, interleukin-1) (Mackay, A. R., et al. 1992, Invasion Metastasis 12:168-184; Lamoreaux, W. J., et al., 1998 Microvascular Res. 55:29-42; Puyrairnond, A, et al., 1999, J. Cell Sci. 112:1283-1290). Many studies have shown that expression and/or increased MMP activity correlate with tumor growth and neovascularization (Johnsen, M., et al., 1998, Curr. Opin. Cell Biol. 10:667-671; Hiraoka, N., et al., 1998, Cell 91:439-442; Brown, P. D., et al., 1993, J. Natl. Cancer Inst. 85:574-578). MMP inhibitors, both synthetic and endogenous, have been used to block angiogenic responses in vitro and in vivo (Murphy, A. N., et al., 1993, J. Cell Physiol. 157:351-358).

In view of the role of angiogenesis in many diseases and disorders, it is desirable to have a means of reducing or inhibiting one or more of the biological effects of this process. It is also desirable to have a means of assaying for the presence of cellular markers for angiogenesis in normal and pathological conditions, and especially cancer.

### SUMMARY OF THE INVENTION

One object of the present invention is to provide a method for inhibiting angiogenesis by supplying to endothelial tissue an effective amount of a PPAR gamma ligand.

Another object of the invention is to provide a method for inhibiting angiogenesis by supplying to endothelial tissue effective amounts of both a PPAR gamma ligand and an RXR receptor ligand.

Another object is to provide a method for treating a tumor, reducing the size of a tumor, reducing the vasculature supporting a tumor or reducing the tumor burden of a mammal by administering to a mammalian patient in need thereof an effective amount of a PPAR gamma ligand, optionally with an RXR receptor ligand.

These and other objects which will become apparent during the course of the following detailed description have been achieved by the present invention. In one aspect, the invention is based on the discovery that PPAR gamma is expressed in endothelial cells. In another aspect, the invention is based on the finding that PPAR gamma ligands inhibit endothelial cell differentiation into new blood vessels. On the basis of these discoveries, the invention is also directed to the use of PPAR gamma ligands to inhibit or reduce tumor or neoplasm growth in a mammal, including a human, in need of such a treatment.

In yet a further aspect, the invention provides an article of manufacture, comprising:

a container;

a label on the container; and

a composition comprising an active agent contained within said container; wherein the label on the container indicates that the composition can be used to inhibit angiogenesis and the active agent in the composition is an PPAR gamma ligand or agonist.

### BRIEF DESCRIPTION OF THE FIGURES

FIGS. 1A, 1B and 1C show the effect of eicosanoid derivatives on HUVEC tube formation in three-dimensional collagen gels.

FIG. 1A shows the formation of an interconnected network of tube-like structures containing lumens (48 hr).

FIG. 1B shows inhibitory effects of 10 μM 15d-PGJ₂ on tube formation. All cells in the gel remain rounded and devoid of vacuoles or lumen like structures.

FIG. 1C shows effects of different eicosanoids on tube formation in three dimensional collagen gels. The drugs were tested at 1, 10 and 100 μM. Data shown are mean±standard error of the mean n≧3. Data are expressed as the percent inhibition compared to controls incubated with growth factors and the vehicle. At the concentrations shown, none of the prostanoids exhibited >10% toxicity based on the XTT assay.

FIGS. 2A and 2B show expression of PPAR in HUVEC.

FIG. 2A shows RT-PCR analysis using the specific primers for PPAR alpha, beta or gamma. Specific complementary DNAs (cDNA) were synthesized from human liver RNA (positive control) and from HUVEC RNA using random primer in the presence of 50 U of Moloney Murine Leukemia virus reverse transcriptase. PCR products were resolved on a 1.5% agarose gel. The DNA ladder was included as a marker to indicate the sizes of the PCR products of PPAR alpha, beta or gamma.

FIG. 2B shows Western blot analysis of PPAR gamma expression in HUVEC. Tissue extract (20 μg protein, positive control) from mouse adipose tissue or HUVEC cell lysates (100 μg protein) prepared from cells cultured as monolayers on type I collagen in 1× basal medium without (lane 1) or containing (lane 2) VEGF (40 ng/ml), bFGF (40 ng/ml) and PMA (80 nM) for 24 hr were immunoprecipitated by PPAR gamma monoclonal antibody. Following SDS-PAGE and transfer to nitrocellulose, the proteins were immunoblotted with the same PPAR gamma monoclonal antibody. Proteins were visualized using the ECL chemiluminescence kit (Amersham, Clearbrook Ill.). Similar results were obtained from two independent experiments with different HUVEC cultures.

FIGS. 3A, 3B and 3C show the effects of PPAR and RXR ligands on tube formation.

FIG. 3A shows dose-dependent inhibition of HTUVEC tube formation by PPAR gamma ligands.

FIG. 3B shows that LY 17883, WY 14643, clofibrate and erucic acid do not inhibit tube formation at 100 μM.

FIG. 3C shows synergistic inhibition of tube formation by the combination of PPAR gamma and RXR specific ligands. Data are expressed as the percent inhibition of control (incubated with growth factors and vehicle). Responses are the mean+standard error (A) or percent control (B and C) for 3-4 independent experiments.

FIGS. 4A, 4B ans 4C show flatmount photographs of rat corneas 5 days after implantation of Hydron pellets containing 200 ng/pellet VEGF (4A) or 200 ng/pellet VEGF and 10 μg/ml 15d-PGJ₂ (4B). Summary data of angiogenic response induced by 200 ng/pellet VEGF or 200 ng/pellet VEGF and 10 [g/ml 15d-PGJ₂. (4C) The area of new vessels was assessed on day 6 postimplantation. Data are expressed as mean±standard error of the mean, * significantly differ from control (p <0.01). + significantly different from VEGF alone group (p<0.05, Mann-Whitney test for non-parametric values).

FIG. 5 shows that PPAR(activation inhibits HUVEC proliferation. HUVEC cultured on type I collagen-coated surfaces in medium containing VEGF, bFGF and PMA were treated with various concentrations of 15d-PGJ₂ or BRL49653. Cell proliferation was monitored as BrdU incorporation. Data are the mean±standard error from 3-4 independent experiments and are expressed as percent inhibition compared to vehicle treated groups in the presence of growth factors.

FIGS. 6A-6D show regulation of angiogenesis associated gene expression by 15d-PGJ₂. Total RNA (100 ng) were isolated from HUVEC grow in three dimensional collagen gels for the indicated times in medium containing VEGF, bFGF and PMA in the presence of 10:M 15d-PGJ₂ (hatched bars) or vehicle (open bars). The mRNA levels of the indicated genes were determined by real time quantitative RT-PCR (Taqman) analysis. The relative expression levels of each gene were normalized to the levels of GAPDH measured in the same RNA preparation. # significantly different from control groups at 4 hr. * significantly different from time-matched control groups. Data are shown as means±standard error from 3-5 independent experiments and were analyzed by one-way ANOVA.

### DEFINITIONS

As used herein, the term “PPAR gamma ligand” or “PPAR gamma agonist” means a compound which binds to and induces at least one biological activity of PPAR gamma. The compound may be a natural or synthetic ligand for the receptor. Any compound which binds to and activates the PPAR gamma may be used in the methods of the invention. Examples of suitable compounds include known ligands such as prostaglandins, thiazolidinediones, non-steroidal anti-inflammatory compounds (NSAIDS), unsaturated fatty acids and receptor binding derivatives thereof Many PPAR gamma binding compounds as well as methods of making or obtaining them are well known in the art. Also included in the scope of the invention are PPAR alpha ligands which, in addition to binding PPAR alpha, also bind PPAR gamma (referred to as PPAR alpha/gamma ligands). It is generally necessary to administer the PPAR alpha/gamma ligands in higher doses to achieve the same level of inhibition of angiogenesis as is obtained when a more specific PPAR gamma ligand is used.

The term “endothelial cell” is used to mean the cells of endothelial tissue. This tissue includes the membranes lining serous cavities, heart, blood and lymph vessels.

A “PPAR gamma” receptor refers to the mammalian PPAR gamma 1 or PPAR gamma 2 isoforms described by Vidal-Pruig et al., ((1997) J. Biol. Chem., 272:8071-8076) together with the naturally occurring allelic and processed forms thereof Preferably, the receptor is “human PPAR gamma”; i.e. having the amino acid sequence of a PPAR gamma from a human source.

“Effective amount” or “therapeutically effective amount” of a PPAR gamma agonist or an RXR receptor ligand/agonist is an amount that is effective either to prevent, lessen the worsening of, alleviate, or cure the treated condition. An example of a effective amount of a PPAR gamma ligand or agonist is an amount of the compound which is sufficient to prevent or reduce angiogenesis in a sample (or patient) relative to a control sample (or patient) in which the ligand is not administered.

“Treatment” refers to both therapeutic treatment and prophylactic or preventative measures. Those in need of treatment include those already with the disorder as well as those in which the disorder is to be prevented.

“Mammal” for purposes of treatment refers to any animal classified as a mammal, including humans, domestic and farm animals, and zoo, sports, or pet animals, such as dogs, horses, cats, cows, etc. Preferably, the mammal is human.

The term “inhibiting” refers to the act of substantially preventing or reducing the development of an event, for example, “inhibiting angiogenesis” means substantially preventing or reducing the development of blood vessels in a treated mammal. Similarly, “inhibiting expression” of an enzyme, such as a MMP, means substantially preventing or reducing the expression of the enzyme by cells which express the enzyme.

The term “neovascularization” refers to growth and development of blood vessels in tissue not normally containing them, or of blood vessels of a different kind than usual in tissue.

“Diseases or disorders characterized by undesirable excessive neovascularization” refer to diseases or disorders that include, by way of example, excessive neovascularization, tumors, and especially solid malignant tumors, rheumatoid arthritis, psoriasis, atherosclerosis, endometriosis, diabetic and other retinopathies, retrolental fibroplasia, age-related macular degeneration, neovascular glaucoma, hemangiomas, thyroid hyperplasias (including Grave's disease), corneal and other tissue transplantation, and chronic inflammation.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemica Approach to Targeted Drug Delivery”, Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, -lactam-containing prodrugs, optionally substituted phenoxyacetamide-containing prodrugs or optionally substituted phenylacetamide-containing prodrugs.

A “liposome” is a small vesicle composed of various types of lipids, phospholipids and/or surfactant which is useful for delivery of a drug (such as the agonists disclosed herein) to a mammal. The components of the liposome are commonly arranged in a bilayer formation, similar to the lipid arrangement of biological membranes.

A “synergistic effect” or a “synergistic manner” is an effect which is achieved by administering two compounds that is greater than the effect of either of the compounds administered individually.

### DETAILED DESCRIPTION OF THE PREFERRED EMBODIMENTS

Angiogenesis is the process of producing new blood vessels. The present invention is based on the discoveries that (1) PPAR gamma is expressed in endothelial tissue (both PPAR gamma 1 and PPAR gamma 2), and (2) activation of PPAR gamma in endothelial tissue using compounds that bind to the receptor, i.e., ligands or agonists of PPAR gamma, inhibit the formation of new blood vessels. Activation of PPAR gamma with a ligand therefor results in potent inhibition of endothelial differentiation into endothelial cell tube like structures which are precursor structures to vessel formation. By reducing vessel formation, the invention reduces the vasculature supporting a tumor, inhibiting tumor size or growth and reducing the tumor burden of the mammal.

PCR is used to detect the expression of all three PPARs (alpha, beta and gamma) mRNAs in the endothelial cells cultured in 3D collagen gels. The expression of PPAR gamma protein expression in endothelial cells was confirmed by Western blots. PPAR gamma protein levels are not significantly altered by the growth factor mixture in the present study. PPAR alpha mRNA expression by HUVEC has been described previously (Inoue, I. et al., (1998) Biocem. Biophys. Res. Commun 246:370-374).

Using an in vitro model of angiogenesis described by Davis et al. (Davis, G., and Camarillo, C. (1996) Exp. Cell Res. 1996 224:39-51) activation of PPAR receptors resulted in inhibition of endothelial tube formation. In this model, endothelial cells are suspended in a three-dimensional collagen lattice of type I collagen and undergo rapid morphogenesis. Within 4 hr numerous vacuoles are observed in the majority of endothelial cells; at 24 hr the formation of tube-like structures can be observed and at 48 hr an interconnected network of tube-like structures is observed. Inhibitors of protein synthesis (cycloheximide) and mRNA synthesis (actinomycin D) completely block this formation of tube-like structures, indicating that there is a requirement for new mRNA and protein synthesis for this formation.

It has also been discovered that angiogenesis is inhibited by the administration of a PPAR gamma ligand/agonist and a retinoic acid (RXR) receptor ligand/agonist in a synergistic manner. In the inhibition of endothelial tube formation assay described above, administration of both types of compounds synergistically inhibits tube formation and angiogenesis to a greater degree than the inhibition caused by either compound alone. The compounds may be administered together or sequentially so long as both compounds are present at inhibitory concentrations and bind the respective receptors at the same time.

The PPAR gamma ligand and the RXR ligand may be administered in any ratio which provides inhibition of angiogenesis. Preferably, the ratio PPAR ligand/RXR ligand will be about 1:1 to about 1:30, more preferably about 1:5 to about 1:20.

Any RXR ligand/agonist may be used in the method of the invention. Suitable RXR ligands are disclosed in U.S. Pat. Nos. 5,824,685; 5,780,676; 5,399,586; 5,466,861; 4,810,804; 5,770,378; 5,770,383; 5,770,382. Examples include 9-cis-retinoic acid, ethyl-6-[2-(4,4-dimethylthiochroman-6-yl)ethynyl]nicotinate, 6-[2-(4,4-dimethylchroman-6-yl)ethynyl]nicotinic acid, p-[(E)-2-(5,6,7,8-tetrahydro-5,5,8,8-tetramethyl-2-naphthyl)propenyl]-benzoic acid, 3-methyl-7-ethyl-9-(2,6,6-trimethyl-1-cyclohexen-1-yl)-2E,4E,6Z,8E-nonatetranoic acid, 3-methyl-7-propyl-9-(2,6,6-trimethyl-1-cyclohexen-1-yl)-2E,4E,6Z,8E-nonatetranoic acid, 3-methyl-7-isopropyl-9-(2,6,6-trimethyl-1-cyclohexen-1-yl)-2E,4E,6Z,8E-nonatetranoic acid, 3,6,7-trimethyl-9-(2,6,6-trimethyl-1-cyclohexen-1-yl)-2E,4E,6Z,8E-nonatetranoic acid, 3-methyl-7-t-butyl-9-(2,6,6-trimethyl-1-cyclohexen-1-yl)-2E,4E,6Z,8E-nonatetranoic acid, 3-methyl-5-{2-[2-(2,6,6-trimethylcyclohexen-1-yl)ethenyl]phenyl}-2E,4E-pentadienoic acid, 3-methyl-5-{2-[2-(2,6,6-trimethylcyclohexen-1-yl)ethenyl]cyclohexyl}-2E,4E-pentadienoic acid, (2E,4E)-3-methyl-6-{ 1-[2,6,6-trimethyl-1-cyclohexenyl)ethenyl]cyclopropyl}-2,4-hexadienoic acid, (2E,4E,6Z)-7-(5,5,8,8-tetramethyl-5,6,7,8-tetrahydro-2-naphthyl)-3,8-dimethyl-nona-2,4,6-trienoic acid, (2E,4E,6Z)-7-(3,5,5,8,8-pentamethyl-5,6,7,8-tetrahydro-2-naphthyl)-3-methyl-octa-2,4,6-trienoic acid, 4-(E)-2-(5,6,7,8-tetrahydro-3,5,5,8,8-pentamethylnaphthalen-2-yl)propen-1-yl]benzoic acid, 4-[4,4-dimethylchroman-6-ylethynyl]benzoic acid, ethyl (2E,4E)-3-methyl-6-(3,4,5,6,7,8-hexahydro-5,5,8,8-tetramethylanthracen-1-yl)hexa-2,4-dienoate; (2E,4E)-3-methyl-6-(3,4,5,6,7,8-hexahydro-5,5,8,8-tetramethylanthracen-1-yl)hexa-2,4-dienoic acid; (3E,5E)-3-methyl-6-(3,4,5,6,7,8-hexahydro-5,5,8,8-tetramethylanthracen-1-yl)hexa-3,5-dienoic acid; ethyl (2E,4E)-3-methyl-6-1,2,3,4,5,6,7,8-octahydro-5,5,8,8-tetramethylanthracen-1-yl)hexa-2,4-dienoate; (2E,4E)-3-methyl-6-(1,2,3,4,5,6,7,8-octahydro-5,5,8,8-tetramethyl-anthracen-1-yl)hexa-2,4-dienoic acid; ethyl (2E,4E)-3-methyl-6-(1,2,3,5,6,7,8-heptahydro-5,5,8,8-tetramethylcyclopenta[b]naphthalen-1-yl)hexa-2,4-dienoate; (2E,4E)-3-methyl-6-1,2,3,(5,6,7,8-heptahydro-5,5,8,8-tetramethyl-cyclopenta[b]-naphthalen-1-yl)hexa-2,4-dienoic acid; ethyl (2E,4E)-3-methyl-6-[(1,2,3,4,7,8,9,10-octahydro-1,1,4,4-tetramethyl-6H-naphthocycloheptan-10-yl]hexa-2,4-dienoate; (2E,4E)-3-methyl-6-[1,2,3,4,7,8,9,10-octahydro-1,1,4,4-tetramethyl-6H-naphtocycloheptan-10-yl]hexa-2,4-dienoic acid; ethyl(2Z,4E)-3-methyl-6-[1,2,3,4,7,8,9,10-octahydro-1,1,4,4-tetramethyl-6H-naphthocycloheptan-10-yl]hexa-2,4-dienoate; (2Z,4E)-3-methyl-6-[1,2,3,4,7,8,9,10-octahhydro-1,1,4,4-tetramethyl-6H-naphthocycloheptan-10-yl]hexa-2,4-dienoic acid; (2Z,4E)-3-methyl-6-[1,2,3,4,7,8,9,10-octahydro-1,1,4,4-tetramethyl-6H-naphthocyclohepta-10-yl]hexa-2,4-dienoic acid; (2Z,4E)-3-methyl-6-[5,6,7,8-tetrahydro-5,5,8,8-tetramethylnaphthyl-(2,3-b)-2,2-dimethylpyran-4-yl]hexa-2,4-dienoic acid; (2E,4E)-3-methyl-6-[5,6,7,8-tetrahydro-5,5,8,8-tetramethylnaphthyl-(2,3-b)-pyran-4-yl]hexa-2,4-dienoic acid; (2E,4E)-3-methyl-6-]5,6,7,8-tetrahydro-5,5,8,8-tetramethylnaphthyl-(2,3-b)-pyran-4-yl]hexa-2,4-dienoic acid; (+)-(2E,4E)-3-methyl-6-(1,2,3,4,5,6,7,8-octahydro-5,5,8,8-tetramethylanthracen-1-yl)hex-2,4-dienoic acid; (−)-(2E,4E)-3-methyl-6-(1,2,3,4,5,6,7,8-octahydro-5,5,8,8-tetramethylanthracen-1-ly)hexa-2,4-dienoic acid; methyl (2E)-3-methyl-6-(1,2,3,4,6,7,8,9-octahydro-6,6,9,9-tetramethylanthracen-1-yl)hexa-2-enoate; (2E)-3-methyl-6-(1,2,3,4,6,7,8,9-octahydro-6,6,9,9-tetramethylanthracen-1-yl)hex-2-enoic acid; ethyl (2E,4E)-3-methyl-6-(7,7,10,10-tetramethyl-2,3,4,5,7,8,9,10-octahydronaphtho[2,3-6]-azepinyl) hexa-2,4-dienoate; (2E,4E)-3-methyl-6-[7,7,10,10-tetramethyl-2,3,4,5,7,8,9,10-octahydronaphtho[2,3-6]azepin-yl)hexa-2,4-dienoic acid; ethyl 3-methyl-6-(3,4,6,7,8,9-hexahydro-6,6,9,9-tetramethyl-2H-benzo [g]quinolin-1-yl)hexa-2,4-dienoate; (2E,4E)-3-methyl-6-[3,4,6,7,8,9-hexahydro-6,6,9,9-tetramethyl-2H-benzo [g]quinolin-1-yl)hexa-2,4-dienoic acid; ethyl (2E,4E)-3-methyl-6-oxo-6-[5,6,7,8-tetrahydro-5,5,8,8-tetramethyl (2,3)naphthyl [b]piperidin-1-yl]hexa-2,4-dienoate; (2E,4E)-3-methyl-6-oxo-6-(3,4,6,7,8,9-hexahydro-6,6,9,9-tetramethyl-2H-benzo[quinolin-1-yl)hexa-2,4-dienoic acid; ethyl (2E,4E)-3-methyl-6-oxo-6-[(2,3,5,6,7,8-hexahydro-5,5,8,8-tetramethyl)benzo[f]indol-1-yl]hexa-2,4-dienoate; (2E,4E)-3-methyl-6-oxo-6[(2,3,5,6,7,8-hexahydro-5,5,8,8-tetramethyl)benzo-[f]-indol-1-yl]hexa-2,4-dienoic acid; ethyl (2E,4E)-3-methyl-6-(2,3,5,6,7,8-hexahydro-5,5,8,8-tetramethylbenzo[f]indol-1-yl]-hexa-2,4-dienoate; (2E,4E)-3-methyl-6-(2,3,5,6,7,8-hexahydro-5,5,8,8-tetramethylbenzo[f]indol-1-yl]hexa-2,4-dienoic acid; (2E,4E)-3-methyl-(6,6,9,9-tetramethyl-2,3,6,9-tetrahydronaphtho[2,3-b]-[1,4]oxazin-4-yl)hexa-2,4-dienoic acid; and (2E,4E)-3-methyl-6-oxo-6-(6,6,9,9-tetrahydro-2,3,6,9-tetrahydronaphtho[2,3-b][1,4]oxazin-4-yl)hexa-2,4-dienoic acid.

Additional RXR ligands can be identified using known screening methods. U.S. Pat. Nos. 5,714,595 and 5,700,682, for example, describe a method for screening retinoid X receptor agonists using a retinoid X receptor expressed by a yeast expression system to screen a compound having a retinoid X receptor agonist activity. Another screen is described for detecting a compound having retinoid X receptor agonist activity by: (1) providing a yeast strain which expresses the retinoic acid receptor and activates a reporter plasmid containing apolipoprotein AI gene site A or a mutated variant thereof; (2) incubating the compound in suitable media and a colorless chromogenic substrate; and (3) examining the media for development of color.

The present invention is not limited to PPAR gamma ligands of any particular chemical class, rather any PPAR gamma ligand may be used in the invention. Suitable assays for determining whether a specific compound is a PPAR gamma ligand include the in vitro assay of Example 1 and the in vivo assay of Example 4 described below. Preferably, the ligands have moderate to high affinity to the receptor, e.g. a Kd (dissociation constant) of 10 pM to 1000 μM, preferably 10 nM to 200 μM, more preferably 30 nM to 100 μM.

In one embodiment, the PPAR gamma ligand for use in the method of the invention is a prostaglandin (PG) having the structure shown below.

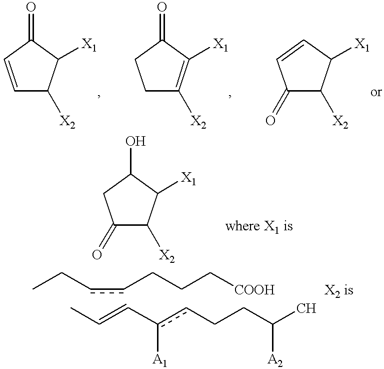

The symbol of a line and a dotted line represents a carbon-carbon double bond or a carbon-carbon single bond. A₁ is H when the symbol of a line and a dotted line represents a carbon-carbon double bond, and is OH when the symbol of a line and a dotted line represents a carbon-carbon single bond, A₂ is H or OH.

Examples include PGA₁, PGA₂, PGB₁, PGB₂, PGD₁, PGD₂, PDJ₂, 15-deoxy-12,14 delta-PGJ₂, and 12-delta-PGJ₂. Since PGD₂ can spontaneously convert to PGJ₂, and in the presence of serum or albumin convert to 15-deoxy-12,14 delta-PGJ₂, it is a naturally occurring endogenous PPAR gamma ligand. These prostanoid compounds are well known in the art.

Suitable ligands also include pro drug forms of the prostaglandins shown above. Examples of prodrugs include hydroxy protected compounds in which an OH group is derivatized to form a hydroxy protecting group, and carboxy protected compounds in which the carboxyl group is derivatized to form a carboxyl protecting group. These protecting goups are well known and can be prepared by known synthetic chemical methods. The prodrugs are generally prepared from the parent compounds using these known reactions and separation techniques.

In another embodiment, the PPAR gamma ligand is an unsaturated fatty acid which binds to PPAR gamma. Preferably, the fatty acid will contain from about 10 to about 26, preferably 18-22, carbon atoms, and contain from zero to about 6, preferably 1,2,3,4,5 or 6, carbon-carbon double bonds, carbon-carbon triple bonds or a combination thereof The carboxylic acid group is generally at a terminal chain position. The double and triple bonds are preferably not conjugated. Examples of suitable fatty acids include palmitic acid (C16:0), oleic acid (C18:0), petroselenic acid (C18:1), linolenic acid (C18:3) arachidonic acid (C20:4), docosahexaenoic acid (DHA; C22:6), parinaric acid and eicosatetraynenoic acid (EHA; C20:4). The fatty acid ligands may bind to both PPAR alpha and PPAR gamma.

In another embodiment, the PPAR gamma ligand is a fibrate capable of activating PPAR gamma A suitable known fibrate has the structure shown below.

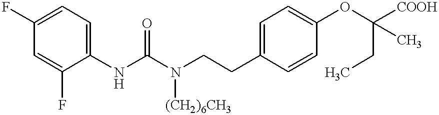

In another embodiment, the PPAR gamma ligand is a thiazolidinedione or a related compound. Many compounds of this class are useful in the method of the present invention, and methods of making these compounds are known. Some of these compounds are disclosed in WO 91/07107; WO 92/02520; WO 94/01433; WO 89/08651; JP Kokai 69383/92; U.S. Pat. Nos. 4,287,200; 4,340,605; 4,438,141; 4,444,779; 4,461,902; 4,572,912; 4,687,777; 4,703,052; 4,725,610; 4,873,255; 4,897,393; 4,897,405; 4,918,091; 4,948,900; 5,002,953; 5,061,717; 5,120,754; 5,132,317; 5,194,443; 5,223,522; 5,232,925; and 5,260,445. The active compounds are disclosed as being useful as therapeutic agents for the treatment of diabetes, hyperglycemia, hypercholesterolemia, and hyperlipidemia. The disclosure of these publications are incorporated herein by reference in particular with respect to the active compounds disclosed therein, and methods of preparation thereof These compounds are also useful for the treatment of angiogenesis and tumor growth in accordance with the present invention. Suitable compounds include thiazolidinedione (TZD) drugs, such as troglitazone, pioglitazone and BRL 49653. Suitable TZD compounds are also described in U.S. Pat. Nos. 5,814,647; 5,811,439; 5,599,826; 5,646,169; and 5,700,820, which are all incorporated herein by reference in their entirety.

Suitable compounds for use in the present invention have formula I

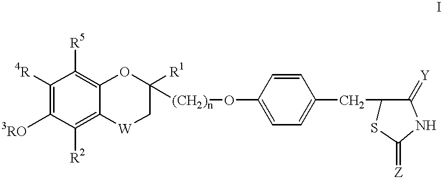

wherein R¹ and R² are the same or different and each is a hydrogen atom or a C₁-C₅ alkyl group;

R³ is a hydrogen atom, a C₁-C₆ aliphatic acyl group, an alicyclic acyl group, an aromatic acyl group, a hetero cyclic acyl group, an araliphatic acyl group, a (C₁-C₆ alkoxy)carbonyl group, or an aralkyloxycarbonyl group;

R⁴ and R⁵ are the same or different and each is a hydrogen atom, a C₁-C₅ alkyl group or a C₁-C₅ alkoxy group, or R⁴ and R⁵ together represent a C₁-C₅ alkylenedioxy group;

n is 1,2, or 3;

W is the —CH₂—, >CO, or CH—OR⁶ group (in which R⁶ is any one of the atoms or groups defined for R³ and may be the same as or different from R³); and

Y and Z are the same or different and each is an oxygen atom or an imino (NH) group;

and pharmaceutically acceptable salts thereof See U.S. Pat. No. 5,478,852, U.S. 5,457,109, and U.S. 5,814,647.

Other suitable compounds have formula II

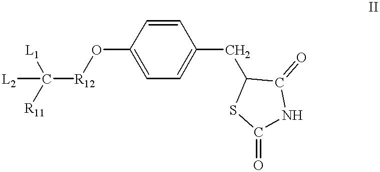

where R₁₁ is substituted or unsubstituted alkyl, alkoxy, cycloalkyl, phenylalkyl, phenyl, aromatic acyl group, a 5- or 6-membered heterocyclic group including 1 or 2 heteroatoms selected from the group consisting of nitrogen, oxygen, and sulfur, or a group of the formula NR₁₃R₄, wherein R₁₃ and R₁₄ are the same or different and each is lower alkyl or R₁₃ and R₁₄ are combined to each other either directly or as interrupted by a heteroatom selected from the group consisting of nitrogen, oxygen, and sulfur to form a 5- or 6-membered ring; wherein R₁₂ means a bond or a lower alkylene group; and wherein L₁ and L₂ are the same or different and each is hydrogen or lower alkyl or L₁ and L₂ are combined to form an alkylene group; or a pharmaceutically acceptable salt thereof See U.S. Pat. No. 5,814,647.

Additional suitable compounds have formula III

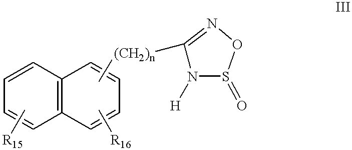

where R₁₅ and R₁₆ are independently hydrogen, lower alkyl containing 1 to 6 carbon atoms, alkoxy containing 1 to 6 carbon atoms, halogen, ethynyl, nitrile, methylthio, trifluoromethyl, vinyl, nitro, or halogen substituted benzyloxy; n is 0 to 4 and the pharmaceutically acceptable salts thereof See U.S. Pat. No. 5,814,647.

The method of the present invention may also use compounds of formula IV

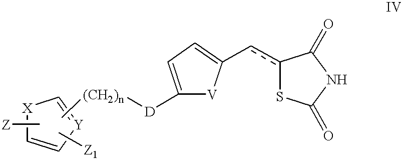

wherein the dotted line represents a bond or no bond;

V is —CH═CH—, —N═CH—, —CH═N— or S;

D is CH₂, CHOH, CO, C═NOR₁₇ or CH═CH;

X is S, O, NR₁₈,—CH═N or —N═CH;

Y is CH or N;

Z is hydrogen, (C₁-C₇)alkyl (C₁-C₇)cycloalkyl, phenyl, naphthyl, pyridyl, furyl, thienyl, or phenyl mono- or disubstituted with the same or different groups which are (C₁-C₃)alkyl, trifluoromethyl, (C₁-C₃)alkoxy, fluoro, chloro, or bromo;

Z₁ is hydrogen or (C₁-C₃)alkyl;

R₁₇ and R₁₈ are each independently hydrogen or methyl; and

n is 1, 2, or 3;

the pharmaceutically acceptable cationic salts thereof; and the pharmaceutically acceptable acid addition salts thereof when the compound contains a basic nitrogen. See U.S. Pat. No. 5,814,647.

The present invention is also directed to the use of compounds of the formula V

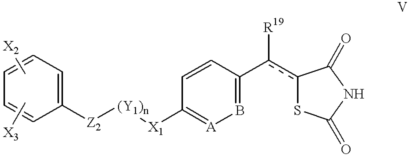

wherein the dotted line represents a bond or no bond;

A and B are each independently CH or N, with the proviso that when A or B is N, the other is CH;

X₁ is S, SO, SO₂, CH₂, CHOH, or CO;

n is 0 or 1;

Y₁ is CHR₂₀ or R₂₁, with the proviso that when n is 1 and

Y₁ is NR₂₁, X₁ is SO₂ or CO;

Z₂ is CHR₂₂, CH₂CH₂, CH═CH,

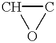

OCH₂, SCH₂, SOCH₂ or SO₂CH₂;

R₁₉, R₂₀, R₂₁, and R₂₂ are each independently hydrogen or methyl; and

X₂ and X₃ are each independently hydrogen, methyl, trifluoromethyl, phenyl, benzyl, hydroxy, methoxy, phenoxy, benzyloxy, bromo, chloro, or fluoro;

a pharmaceutically acceptable cationic salt thereof; or

a pharmaceutically acceptable acid addition salt thereof when A or B is N. See U.S. Pat. No. 5,814,647.

The present invention also relates to the use of compounds of the formula VI

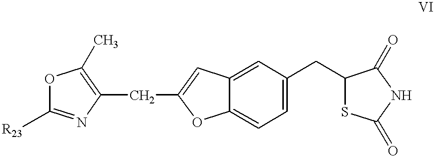

or a pharmaceutically acceptable salt thereof where R₂₃ is alkyl of 1 to 6 carbon atoms, cycloalkyl of 3 to 7 carbon atoms, phenyl or mono- or di-substituted phenyl where the substituents are independently alkyl of 1 to 6 carbon atoms, alkoxy of 1 to 3 carbon atoms, halogen, or trifluoromethyl. See U.S. Pat. No. 5,814,647.

Other suitable compounds have formula VII

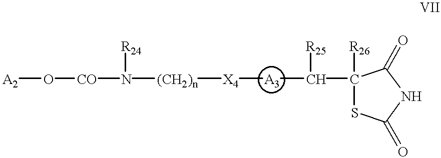

or a tautomeric form thereof and/or a pharmaceutically acceptable salt thereof, and/or a pharmaceutically acceptable solvate thereof, where:

A₂ is an alkyl group, a substituted or unsubstituted aryl group, or an aralkyl group where the alkylene or the aryl moiety may be substituted or unsubstituted;

A₃ is a benzene ring having in total up to 3 optional substituents;

R₂₄ is a hydrogen atom, an alkyl group, an acyl group, an aralkyl group where the alkyl or the aryl moiety may be substituted or unsubstituted, or a substituted or unsubstituted aryl group; or A₂ together with R₂₄ represents substituted or unsubstituted C₂₋₃ polymethylene group, optional substituents for the polymethylene group being selected from alkyl or aryl or adjacent substituents together with the methylene carbon atoms to which they are attached form a substituted or unsubstituted phenylene group;

R₂₅ and R₂₆ each are hydrogen, or R₂₅ and R₂₆ together represent a bond;

X₄ is O or S; and

n is an integer in the range of from 2 to 6. See U.S. Pat. No. 5,814,647.

The present invention may also may also be practiced using a compound of formula VIII

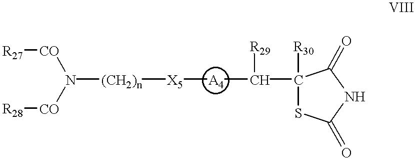

or a tautomeric form thereof and/or a pharmaceutically acceptable salt thereof, and/or a pharmaceutically acceptable solvate therefor, where:

R₂₇ and R₂₈ each independently represent an alkyl group, a substituted or unsubstituted aryl group, or an aralkyl group being substituted or unsubstituted in the aryl or alkyl moiety; or R₂₇ together with R₂, represents a linking group, the linking group consisting of an optionally substituted methylene group and either a further optionally substituted methylene group or an O or S atom, optional substituents for the methylene groups being selected from alkyl-, aryl, or aralkyl, or substituents of adjacent methylene groups together with the carbon atoms to which they are attached form a substituted or unsubstituted phenylene group;

R₂₉ and R₃₀ each represent hydrogen, or R₂₉ and R₃₀ together represent a bond;

A₄ is a benzene ring having in total up to 3 optional substituents; X₅ is O or S; and

n is an integer in the range of from 2 to 6. See U.S. Pat. No. 5,814,647.

The present invention may also may also be practiced using a compound of formula IX

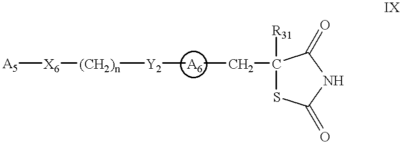

or a tautomeric form thereof and/or a pharmaceutically acceptable salt thereof, and/or a pharmaceutically acceptable solvate thereof, where:

A₅ is a substituted or unsubstituted aromatic heterocyclyl group;

A₆ is a benzene ring having in total up to 5 substituents;

X₆ is O, S, or NR₃₂ where R₃₂ represents a hydrogen atom, an alkyl group, an acyl group, an aralkyl group, wherein the aryl moiety may be substituted or unsubstituted, or a substituted or unsubstituted aryl group;

Y₂ is O or S;

R₃₁ is an alkyl, aralkyl, or aryl group; and

n is an integer in the range of from 2 to 6. See U.S. Pat. No. 5,814,647.

Suitable aromatic heterocyclyl groups include substituted or unsubstituted, single or fused ring aromatic heterocyclyl groups comprising up to 4 hetero atoms in each ring selected from oxygen, sulfur, or nitrogen.

Preferred aromatic heterocyclyl groups include substituted or unsubstituted single ring aromatic heterocyclyl groups having 4 to 7 ring atoms, preferably 5 or 6 ring atoms.

In particular, the aromatic heterocyclyl group contains 1, 2, or 3 heteroatoms, preferably 1 or 2, selected from oxygen, sulfur, or nitrogen.

Suitable groups for A₅ when it represents a 5-membered aromatic heterocyclyl group include thiazolyl and oxazoyl, preferably oxazoyl.

Suitable groups for A₅ when it represents a 6-membered aromatic heterocyclyl group include pyridyl or pyrimidinyl.

R₃₁ is preferably an alkyl group, in particular a C₁₆ alkyl group, for example a methyl group. Preferably, A₅ is a moiety of formula (a), (b), or (c):

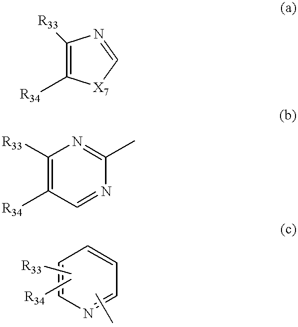

where:

R₃₃ and R₃₄ each independently is a hydrogen atom, an alkyl group, or a substituted or unsubstituted aryl group or when R₃₃ and R₃₄ are each attached to adjacent carbon atoms, then R₃₃ and R₃₄ together with the carbon atoms to which they are attached form a benzene ring wherein each carbon atom represented by R₃₃ and R₃₄ together may be substituted or unsubstituted; and in the moiety of Formula (a), X₇ is oxygen or sulfur.

In one embodiment, R₃₃ and R₃₄ together are a moiety of Formula (d):

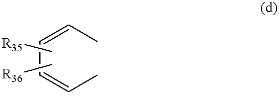

where R₃₅ and R₃₆ each independently are hydrogen, halogen, substituted or unsubstituted alkyl, or alkoxy.

Other suitable compounds have formula X

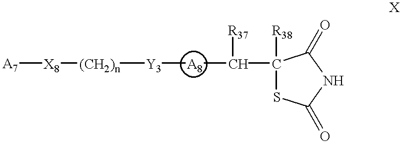

or a tautomeric form thereof and/or a pharmaceutically acceptable salt thereof, and/or a pharmaceutically acceptable solvate thereof, where:

A₇ is a substituted or unsubstituted aryl group;

A, is a benzene ring having in total up to 5 substituents;

X₈ is O, S, or NR₃₉ where R₃₉ is a hydrogen atom, an alkyl group, an acyl group, an aralkyl group, where the aryl moiety may be substituted or unsubstituted, or a substituted or unsubstituted aryl group;

Y₃ is O or S;

R₃₇ is hydrogen;

R₃₈ is hydrogen or an alkyl, aralkyl, or aryl group or R₃₇ together with R₃₈ represents a bond; and n is an integer in the range of from 2 to 6. See U.S. Pat. No. 5,814,647.

Additional compounds have the formula

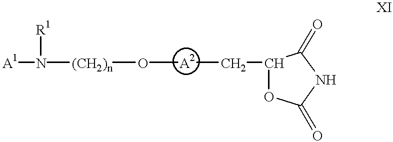

or a tautomeric form thereof and/or a pharmaceutically acceptable salt thereof, and/or a pharmaceutically acceptable solvate thereof, where:

A¹ is a substituted or unsubstituted aromatic heterocyclyl group;

R¹ is a hydrogen atom, an alkyl group, an acyl group, an aralkyl group, where the aryl moiety may be substituted or unsubstituted, or a substituted or unsubstituted aryl group;

A² is a benzene ring having in total up to five substituents; and n is an integer in the range of from 2 to 6.

Suitable aromatic heterocyclyl groups include substituted or unsubstituted, single or fused ring aromatic heterocyclyl groups containing up to 4 heteroatoms in each ring selected from oxygen, sulphur or nitrogen.

Preferred aromatic heterocyclyl groups include substituted or unsubstituted single ring aromatic heterocyclyl groups having 4 to 7 ring atoms, preferably 5 or 6 ring atoms.

In particular, the aromatic heterocyclyl group contains 1, 2 or 3 heteroatoms, especially 1 to 2, selected from oxygen, sulphur or nitrogen.

Suitable groups for A¹ when it is a 5-membered aromatic heterocyclyl group include thiazoylyl and oxazolyl, preferably oxazolyl.

Suitable groups for A¹ when it is a 6-membered aromatic heterocyclyl group include pyridyl or pyrimidinyl.

Preferably, A¹ is a moiety of formula (a), (b) or (c):

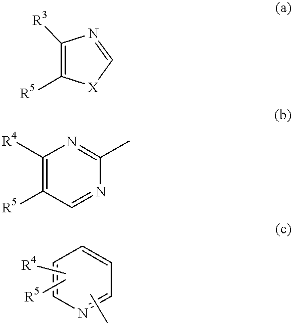

where:

R⁴ and R⁵ each independently is a hydrogen atom, an alkyl group or a substituted or unsubstituted aryl group or when R⁴ and R⁵ are each attached to adjacent carbon atoms, then R⁴ and R⁵ together with the carbon atoms to which they are attached form a benzene ring wherein each carbon atom represented by R⁴ and R⁵ together may be substituted or unsubstituted; and in the moiety of formula (a) X is oxygen or sulfur.

Other compounds useful in the present invention have the formulas

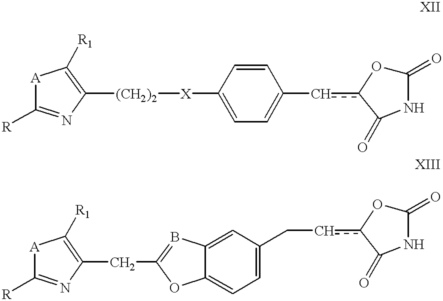

or a pharmaceutically acceptable salt thereof where the dotted fine represents a bond or no bond; R is cycloalkyl of three to seven carbon atoms, naphthyl, thienyl, furyl, phenyl or substituted phenyl where the substituent is alkyl of one to three carbon atoms, alkoxy of one to three carbon atoms, trifluoromethyl, chloro, fluoro or bis(trifluoromethyl); R₁ is alkyl of one to three carbon atoms; X is O or CO; A is O or S; and B is N of CH.

A preferred group of compounds are those of formula XI where the dotted line represents no bond, R₁ is methyl, X is O and A is O. Especially preferred within this group are the compounds where R₁ is phenyl, 2-naphthyl and 3,5-bis(trifluoromethyl)phenyl.

A second group of preferred compounds are those of formula XII where the dotted line represents no bond, R₁ is methyl and A is O. Especially preferred within this group are compounds where B is CH and R is phenol, p-tolyl, m-tolyl, cyclohexyl and 2-naphthyl. Also especially preferred is the compound where B is N and R is phenyl.

A still further embodiment of the present invention is the use of a pharmaceutical composition for administering an effective amount of a compound of the preceding Formulas I through XIII along with a pharmaceutically acceptable carrier in unit dosage form in the method of the invention.

The compounds used in the treatment methods of the invention, which are 5-[4-(chromoanalkoxy)benzyl]-thiazolidene derivatives, may be represented by the Formulas (Ia), (Ib), and (Ic)

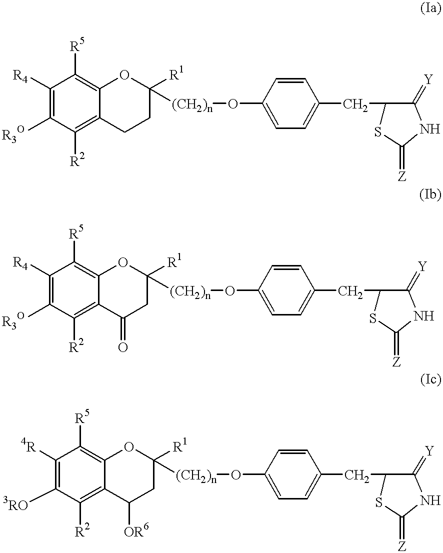

(in which R¹, R², R³, R⁴, R⁵, R⁶, n, Y, and Z are as defined above) and include pharmaceutically acceptable salts thereof:

In the compounds of the invention where R¹ or R² represents an alkyl group, this may be a straight or branched chain alkyl group having from 1 to 5 carbon atoms and is preferably a primary or secondary alkyl group, for example the methyl, ethyl, propyl, isopropyl, butyl, isobutyl, pentyl, or isopentyl group.

Where R³, R⁶, or R^{6′} is an aliphatic acyl group, this preferably has from 1 to 6 carbon atoms and may include one or more carbon-carbon double or triple bonds. Examples of such groups include the formyl, acetyl, propionyl, butyryl, isobutyryl, pivaloyl, hexanoyl, acryloyl, methacryloyl, and crotonyl groups.

Where R³, R⁶, or R^{6′} is an alicyclic acyl group, it is preferably a cyclopentanecarbonyl, cyclohexanecarbonyl, or cycloheptanecarbonyl group.

Where R³, R⁶, or R^{6′} is an aromatic acyl group, the aromatic moiety thereof may optionally have one or more substituents (for example, nitro, amino, alkylamino, dialkylamino, alkoxy, halo, alkyl, or hydroxy substituents); examples of such aromatic acyl groups included the benzoyl, p-nitrobenzoyl, m-fluorobenzoyl, o-chlorobenzoyl, p-aminobenzoyl, m-(dimethylamino)benzoyl, o-methoxybenzoyl, 3,4-dichlorobenzoyl, 3,5-di-t-butyl-4-hydroxybenzoyl, and 1-naphthoyl groups.

Where R³, R⁶, or R^{6′} is a heterocyclic acyl group, the heterocyclic moiety thereof preferably has one or more, preferably one, oxygen, sulfur, or nitrogen hetero atoms and has from 4 to 7 ring atoms; examples of such heterocyclic acyl groups include the 2-furoyl, 3-thenoyl, 3-pyridinecarbonyl (nicotinoyl), and 4-pyridinecarbonyl groups.

Where R³, R⁶, or R^{6′} is an araliphatic acyl group, the aliphatic moiety thereof may optionally have one or more carbon-carbon double or triple bonds and the aryl moiety thereof may optionally have one or more substituents (for example, nitro, amino, alkylamino, dialkylamino, alkoxy, halo, alkyl, or hydroxy substituents); examples of such araliphatic acyl groups include the phenylacetyl, p-chlorophenylacetyl, phenylpropionyl, and cinnamoyl groups.

Where R³, R⁶, or R^{6′} is a (C₁-C₆ alkoxy)carbonyl group, the alkyl moiety thereof may be any one of those alkyl groups as defined for R¹ and R², but is preferably a methyl or ethyl group, and the alkoxycarbonyl group represented by R³, R⁶, or R^{6′} is preferably a methoxycarbonyl or ethoxycarbonyl group.

Where R³, R⁶, or R^{6′} is an aralkyloxycarbonyl group, the aralkyl moiety thereof may be any one of those included within the araliphatic acyl group represented by R³, R⁶, or R^{6′}, but is preferably a benzyloxycarbonyl group.

Where R⁴ and R⁵ are alkyl groups, they may be the same or different and may be straight or branched chain alkyl groups. They preferably have from 1 to 5 carbon atoms and examples include the methyl, ethyl, propyl, isopropyl, butyl, isobutyl, t-butyl, pentyl, and isopentyl groups.

Where R⁴ and R⁵ are alkoxy groups, these may be the same or different and may be straight or branched chain groups, preferably having from 1 to 4 carbon atoms. Examples include the methoxy, ethoxy, propoxy, isopropoxy, and butoxy groups. Alternatively, R⁴ and R⁵ may together represent a C₁-C₄ alkylenedioxy group, more preferably a methylenedioxy or ethylenedioxy group.

Preferred classes of compounds of Formula I are as follows:

(1) Compounds in which R³ is a hydrogen atom, a C₁-C₆ aliphatic acyl group, an aromatic acyl group, or a heterocyclic acyl group.

(2) Compounds in which Y is an oxygen atom; R¹ and R² are the same or different and each is a hydrogen atom or a C₁-C₅ alkyl group; R³ is a hydrogen atom, a C₁-C₆ aliphatic acyl group, an aromatic acyl group, or a pyridinecarbonyl group; and R⁴ and R⁵ are the same or different and each is a hydrogen atom, a C₁-C₅ alkyl group, or a C₁ or C₂ alkoxy group.

(3) Compounds as defined in (2) above, in which: R¹, R², R⁴, and R⁵ are the same or different and each is a hydrogen atom or a C₁-C₅ alkyl group; n is 1 or 2; and W is the —CH₂— or >CO group.

(4) Compounds as defined in (3) above, in which R³ is a hydrogen atom, a C₁-C₅ aliphatic acyl group, a benzoyl group, or a nicotinyl group.

(5) Compounds as defined in (4) above, in which: R¹ and R⁴ are the same or different and each is a C₁-C₅ alkyl group; R² and R⁵ are the same or different and each is the hydrogen atom or the methyl group; and R³ is a hydrogen atom or a C₁-C₄ aliphatic acyl group.

(6) Compounds in which: W is the —CH₂— or >CO group; Y and Z both represent oxygen atoms; n is 1 or 2; R¹ and R⁴ are the same or different and each is a C₁-C₄ alkyl group; R² and R⁵ are the same or different and each is the hydrogen atom or the methyl group; and R³ is a hydrogen atom or a C₁-C₄ aliphatic acyl group.

(7) Compounds as defined in (6) above, in which n is 1.

(8) Compounds as defined in (6) or (7) above, in which W is the —CH₂— group.

Preferred compounds among the compounds of Formula I are those wherein:

R¹ is a C₁-C₄ alkyl group, more preferably a methyl or isobutyl group, most preferably a methyl group;

R² is a hydrogen atom or a C₁-C₄ alkyl group, preferably a hydrogen atom, or a methyl or isopropyl group, more preferably a hydrogen atom or a methyl group, most preferably a methyl group;

R³ is a hydrogen atom, a C₁-C₄ aliphatic acyl group, an aromatic acyl group or a pyridinecarbonyl group, preferably a hydrogen atom, or an acetyl, butyryl, benzoyl, or nicotinyl group, more preferably a hydrogen atom or an acetyl, butyryl or benzoyl group, most preferably a hydrogen atom or an acetyl group;

R⁴ is a hydrogen atom, a C₁-C₄ alkyl group or a C₁ or C₂ alkoxy group, preferably a methyl, isopropyl, t-butyl, or methoxy group, more preferably a methyl or t-butyl group, most preferably a methyl group;

R⁵ is a hydrogen atom, a C₁-C₄ alkyl group or a C₁ or C₂ alkoxy group, preferably a hydrogen atom, or a methyl or methoxy group, more preferably a hydrogen atom or a methyl group, and most preferably a methyl group;

n is 1 or 2, preferably 1;

Y is an oxygen atom;

Z is an oxygen atom or an imino group, most preferably an oxygen atom; and

W is a —CH₂— or >CO group, preferably a —CH₂ group.

Referring to the general Formula II, the substituents may be any from 1 to 3 selected from nitro, amino, alkylamino, dialkylamino, alkoxy, halo, alkyl, or hydroxy, the aromatic acyl group may be benzoyl and naphthoyl. The alkyl group R₁₁ may be a straight chain or branched alkyl of 1 to 10 carbon atoms, such as methyl, ethyl, n-propyl, i-propyl n-butyl, i-butyl, t-butyl, n-pentyl, i-pentyl, n-hexyl, n-heptyl, n-octyl, n-nonyl, and n-decyl; the cycloalkyl group R₁₁ may be a cycloalkyl group of 3 to 7 carbon atoms, such as cyclopropyl, cyclopentyl, cyclohexyl, and cycloheptyl; and the phenylalkyl group R₁₁ may be a phenylalkyl group of 7 to 11 carbon atoms such as benzyl and phenethyl. As examples of the heterocyclic group R₁₁ may be mentioned 5- or 6-membered groups each including 1 or 2 hetero-atoms selected from among nitrogen, oxygen, and sulfur, such as pyridyl, thienyl furyl, thiazolyl, etc. When R₁₁ is NR₁₃R₁₄, the lower alkyls R₁₃ and R₁₄ may each be a lower alkyl of 1 to 4 carbon atoms, such as methyl, ethyl, n-propyl, i-propyl, and n-butyl. When R₁₃ and R₁₄ are combined to each other to form a 5- or 6-membered heterocyclic group as taken together with the adjacent N atom, i.e., in the form of

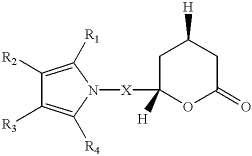

this heterocyclic group may further include a heteroatom selected from among nitrogen, oxygen, and sulfur as exemplified by piperidino, morpholino, pyrrolidino, and piperazino. The lower alkylene group R₁₂ may contain 1 to 3 carbon atoms and thus may be, for example, methylene, ethylene, or trimethylene. The bond R₁₂ is equivalent to the symbol “—”, “.”, or the like which is used in chemical structural formulas, and when R₁₂ represents such a bond, the compound of general Formula II is represented by the following general formula II(a)

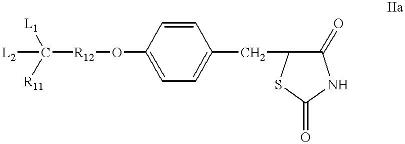

Thus, when R₁₂ is a bond, the atoms adjacent thereto on both sides are directly combined together. As examples of the lower alkyls L₁ and L₂, there may be mentioned lower alkyl groups of 1 to 3 carbon atoms, such as methyl and ethyl. The alkylene group formed as L₁ and L₂ are joined together is a group of the formula —(CH₂)— (where n is an integer of 2 to 6). The cycloalkyl, phenylalkyl, phenyl, and heterocyclic groups mentioned above, as well as the heterocyclic group

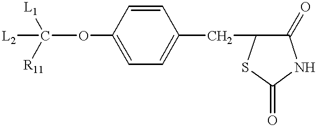

may have 1 to 3 substituents in optional positions on the respective rings. As examples of such substituents may be mentioned lower alkyls (e.g., methyl, ethyl, etc.), lower alkoxy groups (e.g., methoxy, ethoxy, etc.), halogens (e.g., chlorine, bromine, etc.), and hydroxyl. The case also falls within the scope of the general Formula II that an alkylenedioxy group of the formula —O—(CH₂)ₘ—O— (wherein m is an integer of 1 to 3), such as methylenedioxy, is attached to the two adjacent carbon atoms on the ring to form an additional ring.

The preferred compounds of Formula III are those wherein R₁₅ and R₁₆ are independently hydrogen, lower alkyl containing 1 to 6 carbon atoms, alkoxy containing 1 to 6 carbon atoms, halogen, ethynyl, nitrile, trifluoromethyl, vinyl, or nitro; n is 1 or 2 and the pharmaceutically acceptable salts thereof.

Preferred in Formula IV are compounds wherein the dotted line represents no bond, particularly wherein D is CO or CHOH. More preferred are compounds wherein V is —CH═CH—, —CH═N— or S and n is 2, particularly those compounds wherein X is O and Y is N, X is S and Y is N, X is S and Y is CH or X is —CH═N— and Y is CH. In the most preferred compounds X is O or S and Y is N forming an oxazol-4-yl, oxazol-5-yl, thiazol-4-yl, or thiazol-5-yl group; most particularly a 2-[(2-thienyl), (2-furyl), phenyl, or substituted phenyl]-5-methyl-4-oxazolyl group.

The preferred compounds in Formula V are:

a) those wherein the dotted line represents no bond, A and B are each CH, X₁ is CO, n is O, R₁₉ is hydrogen, Z₂ is CH₂CH₂ or CH═CH and X₃ is hydrogen, particularly when X₂ is hydrogen, 2-methoxy, 4-benzyloxy, or 4-phenyl;

b) those wherein A and B are each CH, X₁ is S or SO₂, n is O, R₁₉ is hydrogen, Z₂ is CH₂CH₂ and X₃ is hydrogen, particularly when X₂ is hydrogen or 4-chloro.

A preferred group of compounds is that of Formula VI wherein R₂₃ is (C,-C₆)alkyl, (C₃-C₇)cycloalkyl, phenyl, halophenyl, or (C₁-C₆)alkylphenyl. Especially preferred within this group are the compounds where R₂₃ is phenyl, methylphenyl, fluorophenyl, chlorophenyl, or cyclohexyl.

When used herein with regard to Formulas VII through X, the term “aryl” includes phenyl and naphthyl, suitably phenyl, optionally substituted with up to 5, preferably up to 3, groups selected from halogen, alkyl, phenyl, alkoxy, haloalkyl, hydroxy, amino, nitro, carboxy, alkoxycarbonyl, alkoxycarbonylalkyl, alkylcarbonyloxy, or alkylcarbonyl groups.

The term “halogen” refers to fluorine, chlorine, bromine, and iodine; preferably chlorine.

The terms “alkyl” and “alkoxy” relate to groups having straight or branched carbon chains, containing up to 12 carbon atoms.

Suitable alkyl groups are C₁₋₁₂ alkyl groups, especially C₁₋₆ alkyl groups, e.g., methyl, ethyl, n-propyl, iso-propyl, n-butyl, isobutyl, or tert-butyl groups.

Suitable substituents for any alkyl group include those indicated above in relation to the term “aryl”.

Suitable substituents for any heterocyclyl group include up to 4 substituents selected from the group consisting of alkyl, alkoxy, aryl, and halogen or any 2 substituents on adjacent carbon atoms, together with the carbon atoms to which they are attached, may form an aryl group, preferably a benzene ring, and wherein the carbon atoms of the aryl group represented by the 2 substituents may themselves be substituted or unsubstituted.

Specific compounds include: (+)-5-[[4-[(3,4-dihydro-6-hydroxy-2,5,7,8-tetramethyl-2H-1-benzopyran-2-yl)methoxy]phenyl]methyl]-2,4-thiazolidinedione: (troglitazone);

5-[4-[2-(5-ethylpyridin-2-yl)ethoxyl]benzyl]thiadiazolidine-2,4-dione: (pioglitazone);

5-[4-[(1-methylcyclohexyl)methoxy]benzyl]thiadiazolidine-2,4-dione: (ciglitazone);

4-(2-naphthylmethyl)-1,2,3,5-oxathiadiazole-2-oxide;

5-[4-[2-[N-(benzoxazol-2-yl)-N-methylamino]ethoxy]benzyl]-5-methylthiazolidine-2,4-di one;

5-[4-[2-[2,4-dioxo-5-phenylthiazolidin-3-yl)ethoxy]benzyl]thiazolidine-2,4-dione;

5-[4-[2[N-methyl-N-(phenoxycarbonyl)amino]ethoxy]benzyl]thiazolidine-2,4-dione;

5-[4-[2-phenoxyethoxy)benzyl]thiazolidine-2,4-dione;

5-[4-[2-(4-chlorophenyl)ethylsulfonyl]benzyl]thiazolidine-2,4-dione;

5-[4-[3-(5-methyl-2-phenyloxazol-4-yl)propionyl]benzyl]thiazolidine-2,4-dione;

5-[[4-(3-hydroxy-1-methylcyclohexyl)methoxy]benzyl]thiadiazolidine-2,4-dione;

5-[4-[2-(5-methyl-2-phenyloxazol-4-yl)ethoxyl]benzyl]thiadiazolidione-2,4-dione;

5-[(2-benzyl-2,3-dihydrobenzopyran)-5-ylmethyl]thiadiazoline-2,4-dione: (englitazone);

5-[[2-(2-naphthylmethyl)benzoxazol]-5-ylmethyl]thiadiazoline-2,4-dione;

5-[4-[2-(3-phenylureido)ethoxyl]benzyl]thiadiazoline-2,4-dione;

5-[4-[2-[N-(benzoxazol-2-yl)-N-methylamino]ethoxy]benzy]thiadiazoline-2,4-dione;

5-[4-[3-(5-methyl-2-phenyloxazol-4-yl)propionyl]benzyl]thiadiazoline-2,4-dione;

5-[2-(5-methyl-2-phenyloxazol-4-ylmethyl)benzofuran-5-ylmethyl]-oxazolidine-2,4-dione;

5-[4-[2-[N-methyl-N-(2-pyridyl)amino]ethoxy]benzyl]thiazolidine-2,4-dione (BRL 49653); and

5-[4-[2-[N-(benzoxazol-2-yl)-N-methylamino]ethoxy]benzyl]-oxazolidine-2,4-dione.

Other suitable PPAR receptor ligands are non-steroidal anti-inflammatory drugs including indomethacin and ibuprofen.

Pharmaceutical compositions can be prepared from the compounds of the present invention in conventional formulations. Pharmaceutically acceptable carriers can be either solid or liquid. Solid preparations include powders, tablets, pills, capsules, cachets, suppositories, and dispersible granules. A solid carrier can be one or more substances which may also act as diluents, flavoring agents, binders, preservatives, tablet disintegrating agents, or encapsulating materials.

In powders, the carrier is a finely divided solid mixed with a finely divided active component. In tablets, the active component is mixed with a carrier having the necessary binding properties in suitable proportions and compacted in the size and shape desired. The powders and tablets preferably contain from 5% or 10% to about 70% of the active compound. Suitable carriers are magnesium carbonate, magnesium stearate, talc, sugar, lactose, pectin, dextrin, starch, gelatin, tragacanth, methylcellulose, sodium carboxymethylcellulose, low melting waxes, cocoa butter and the like. The term “preparation” includes formulation of the active compound with encapsulating material as a carrier, providing a capsule in which the active component, with or without other carriers, is surrounded by a carrier and in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active compound is dispersed homogeneously therein, as by stirring. The molten homogenous mixture is then poured into convenient size molds, allowed to cool and solidify.

Liquid preparations include solutions, suspensions and emulsions; for example, water or water/propylene glycol solutions. For parenteral injection, liquid preparations can be formulated in aqueous polyethylene glycol solution.

Aqueous solutions suitable for oral use can be prepared by dissolving the active component in water and adding suitable colorants, flavors, stabilizing and thickening agents as desired.

Aqueous suspensions suitable for oral use can be made by dispersing the finely divided active component in water with a viscous material, such as natural or synthetic gums, resins, methylcellulose, sodium carboxymethylcellulose, and other well-known suspension agents.

Also included are solid preparations which are intended to be converted shortly before use to liquid preparations for oral administration. Such liquid forms include solutions, suspensions and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form In such form the preparation is subdivided into unit doses containing appropriate quantities of the active component. The unit dosage form can be a packaged preparation, the package containing discrete quantities of preparation, such as packeted tablets, capsules, and powders in vials or ampules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 2000 mg, preferably 0.5 mg to 100 mg, according to the application and potency of the active component. The composition can also contain other compatible therapeutic agents.

Many compounds of the present invention are capable of forming pharmaceutically acceptable acid addition and/or base salts. All of these forms are within the scope of the invention. Pharmaceutically acceptable acid addition salts include salts derived from non-toxic inorganic acids, such as hydrochloric, nitric, phosphoric, sulfuric, hydrobromic, hydriodic, hydrofluoric, phosphorous, and the like, as well as the salts derived from non-toxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoracetate, propionate, caprylate, isobutyrate, oxalate, malonate, succinate, suberate, sebacate, fumarate, malate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate, n-methyl glucamine.

The acid addition salts of said basic compounds are prepared by contacting the free base form with a sufficient amount of the desired acid to produce the salt in a conventional manner. The free base form may be regenerated by contacting the salt with a base in the conventional manner. The free base forms differ from their respective salt forms somewhat in certain physical properties, such as solubility in polar solvents, but otherwise the salts are equivalent to the respective free base for purposes of the present invention.

Pharmaceutically acceptable base addition salts are formed with metals or amines, such as alkali and alkaline earth metals or organic amines. Examples of metals used as cations are sodium, potassium, lithium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine.

The base addition salts of said acidic compounds are prepared by contacting the free acid form with a sufficient amount of the desired base to produce the salt in the conventional manner. The free acid form may be regenerated by contacting the salt with an acid and isolating the free acid in the conventional manner. The free acid forms differ from their respective salt forms somewhat in certain physical properties, such as solubility in polar solvents, but otherwise the salts are equivalent to the respective free acids for purposes of the present invention.

Certain of the compounds of the present invention can exist in unsolvated forms as well as solvated forms, including hydrated forms. In general, these forms are equivalent to the unsolvated forms and are within the scope of the present invention. Certain of the compounds of the present invention possess one or more chiral centers. The compounds can, therefore, form steriosomers and diastereomers. The present invention includes the use of both the individual, isolated steriosomers, and mixtures thereof including racemates and partially resolved mixtures. Where stereospecific synthetic techniques are employed or optically active compounds are employed as starting materials in the preparation of the compounds, individual isomers may be prepared directly. Alternatively, if a mixture of isomers is prepared, the individual isomers may be obtained by conventional resolution techniques, or the mixture may be used without optical resolution.

Suitable dosage forms encompass pharmaceutically acceptable carriers that are inherently nontoxic and nontherapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol. Carriers for topical or gel-based forms of antagonist include polysaccharides such as sodium carboxymethylcellulose or methylcellulose, polyvinylpyrrolidone, polyacrylates, polyoxyethylene-polyoxypropylene-block polymers, polyethylene glycol, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations. The agonist will typically be formulated in such vehicles at a concentration of about 0.1 mg/ml to 100 mg/ml.

Suitable examples of sustained release preparations include semipermeable matrices of solid hydrophobic polymers containing the agonist, which matrices are in the form of shaped articles, e.g. films, or microcapsules. Examples of sustained-release matrices include polyesters, hydrogels (for example, poly(2-hydroxyethyl-methacrylate) as described by Langer et al., J. Biomed. Mater. Res. 15:167 (1981) and Langer, Chemr Tech., 12: 98-105 (1982), or poly(vinylalcohol), polylactides (U.S. Pat. No. 3,773,919), copolymers of L-glutamic acid and gamma ethyl-L-glutamate (Sidman et al., Biopolymers, 22:547 (1983), non-degradable ethylene-vinyl acetate (Langer et a., supra), degradable lactic acid-glycolic acid copolymers such as the Lupron Depot™ (injectable micropheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release for shorter time periods.

Sustained-release agonist compositions also include liposomally entrapped compounds. Liposomes containing the agonists are prepared by methods known in the art, such as described in Epstein, et al., Proc. Natl. Acad. Sci. USA, 82:3688 (1985); Hwang, et al., Proc. Natl. Acad. Sci. USA, 77:4030 (1980); U.S. Pat. No. 4,485,045; U.S. Patent No. 4,544,545. Ordinarily the liposomes are the small (about 200-800 Angstroms) unilamelar type in which the lipid content is greater than about 30 mol. % cholesterol, the selected proportion being adjusted for the optimal HRG therapy. Liposomes with enhanced circulation time are disclosed in U.S. Pat. No. 5,013,556.

Therapeutic Uses

For therapeutic applications, the PPAR gamma and optionally the RXR receptor agonists/ligands are administered to a mammal, preferably a human, in a pharmaceutically acceptable dosage form, including those that may be administered to a human intravenously as a bolus or by continuous infusion over a period of time, by intramuscular, intraperitoneal, intra-cerobrospinal, subcutaneous, intra-articular, intrasynovial, intrathecal, oral, topical, or inhalation routes. The agonists also are suitably administered by intratumoral, peritumoral, intralesional, intraocular or perilesional routes, to exert local as well as systemic therapeutic effects.

For the prevention or treatment of disease, the appropriate dosage of agonist will depend on the type of disease to be treated, as defined above, the severity and course of the disease, whether the antibodies are administered for preventive or therapeutic purposes, previous therapy, the patient's clinical history and response to the agonist, and the discretion of the attending physician. The PPAR and optionally the RXR receptor agonist is suitably administered to the patient at one time or over a series of treatments.

The PPAR gamma and optionally the RXR receptor agonists are useful in the treatment of various neoplastic and non-neoplastic diseases and disorders. Neoplasms and related conditions that are amenable to treatment include breast carcinomas, lung carcinomas, gastric carcinomas, esophageal carcinomas, colorectal carcinomas, liver carcinomas, ovarian carcinomas, thecomas, arrhenoblastomas, cervical carcinomas, endometrial carcinoma, endometrial hyperplasia, endometriosis, fibrosarcomas, choriocarcinoma, head and neck cancer, nasopharyngeal carcinoma, laryngeal carcinomas, hepatoblastoma, Kaposi's sarcoma, melanoma, skin carcinomas, hemangioma, cavernous hemangioma, hemangioblastoma, pancreas carcinomas, retinoblastoma, astrocytoma, glioblastoma, Schwannoma, oligodendroglioma, medulloblastoma, neuroblastomas, rhabdomyosarcoma, osteogenic sarcoma, leiomyosarcomas, urinary tract carcinomas, thyroid carcinomas, Wilm's tumor, renal cell carcinoma, prostate carcinoma, abnormal vascular proliferation associated with phakomatoses, edema (such as that associated with brain tumors), and Meigs' syndrome. Excessive endometrial angiogenesis has been reported as involved in the pathogenesis of endometriosis (Healy, D. L., et al., 1998, Hum. Reprod. Update 4:736-740).

In one embodiment of the method of the invention, a PPAR gamma ligand is administered to a mammal, e.g. a human patient, in need thereof to reduce the tumor burden in the mammal. The ligands inhibit angiogenesis and thus are useful for the treatment of diseases or disorders characterized by undesirable excessive neovascularization, including by way of example tumors, and especially solid malignant tumors, rheumatoid arthritis, psoriasis, atherosclerosis, diabetic and other retinopathies, retrolental fibroplasia, age-related macular degeneration, neovascular glaucoma, hemangiomas, thyroid hyperplasias (including Grave's disease), corneal and other tissue transplantation, lung inflammation, obesity and chronic inflammation.

Age-related macular degeneration (AMD) is a leading cause of severe visual loss in the elderly population. The exudative form of AMD is characterized by choroidal neovascularization and retinal pigment epithelial cell detachment. Because choroidal neovascularization is associated with a dramatic worsening in prognosis, the agonists of the present invention are expected to be especially useful in reducing the severity of AMD.

Depending on the type and severity of the disease, about 1 μg/kg to 15 mg/kg of agonist is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is repeated until a desired suppression of disease symptoms occurs. However, other dosage regimens may be useful. The progress of this therapy is easily monitored by conventional techniques and assays, including, for example, radiographic tumor imaging.

According to another embodiment of the invention, the effectiveness of the agonist in preventing or treating disease may be improved by administering the agonist serially or in combination with another agent that is effective for those purposes, such as immunoadhesins, ribozymes, antisense agents, tumor necrosis factor (TNF), an antibody capable of inhibiting or neutralizing the angiogenic activity of acidic or basic fibroblast growth factor (FGF), vascular endothelial growth factor (VEGF), or hepatocyte growth factor (HGF), an antibody capable of inhibiting or neutralizing the coagulant activities of tissue factor, protein C, or protein S (see Esmon, et al., PCT Patent Publication No. WO 91/01753, published Feb. 21, 1991), an antibody capable of binding to HER2 receptor (see Hudziak, et al., PCT Patent Publication No. WO 89/06692, published Jul. 27, 1989), or one or more conventional therapeutic agents such as, for example, alkylating agents, folic acid antagonists, anti-metabolites of nucleic acid metabolism, antibiotics, pyrimidine analogs, 5-fluorouracil, cisplatin, purine nucleosides, amines, amino acids, triazol nucleosides, corticosteroids and proteins such as angiostatin, endostatin, thrombospondin, and platelet factor 4. Such other agents may be present in the composition being administered or may be administered separately. Also, the antagonist is suitably administered serially or in combination with radiological treatments, whether involving irradiation or administration of radioactive substances.

In one embodiment, vascularization of tumors is attacked in combination therapy. One or more PPAR gamma agonists are administered to tumor-bearing patients at therapeutically effective doses as determined for example by observing necrosis of the tumor or its metastatic foci, if any. This therapy is continued until such time as no further beneficial effect is observed or clinical examination shows no trace of the tumor or any metastatic foci. Then TNF is administered, alone or in combination with an auxiliary agent such as alpha-, beta-, or gamma-interferon, anti-HER2 antibody, heregulin, anti-heregulin antibody, D-factor, interleukin-1 (IL-1), interleukin-2 (IL-2), granulocyte-macrophage colony stimulating factor (GM-CSF), or agents that promote microvascular coagulation in tumors, such as anti-protein C antibody, anti-protein S antibody, or C4b binding protein (see Esmon, et al., PCT Patent Publication No. WO 91/01753, published Feb. 21, 1991), or heat or radiation.

Since the auxiliary agents will vary in their effectiveness it is desireable to compare their impact on the tumor by matrix screening in conventional fashion. The administration of PPAR gamma agonist and TNF is repeated until the desired clinical effect is achieved. Alternatively, the PPAR gamma agonist(s) are administered together with TNF and, optionally, auxiliary agent(s). In instances where solid tumors are found in the limbs or in other locations susceptible to isolation from the general circulation, the therapeutic agents described herein are administered to the isolated tumor or organ. In other embodiments, a FGF or platelet-derived growth factor (PDGF) antagonist, such as an anti-FGF or an anti-PDGF neutralizing antibody, is administered to the patient in conjunction with the PPAR agonist. Treatment with PPAR gamma agonists optimally may be suspended during periods of wound healing or desirable neovascularization.

In the uses described above, the PPAR gamma ligand may be combined with an RXR agonist.

Another use of the present invention comprises incorporating a PPAR gamma ligand and optionally an RXR agonist into formed articles. Such articles can be used in modulating endothelial cell growth and angiogenesis. In addition, tumor invasion and metastasis may be modulated with these articles. The ligands may also be placed in a container having a label on the container, where the label on the container indicates that the composition can be used to inhibit angiogenesis and the active agent in the composition is an PPAR gamma, and optionally an RXR ligand or agonist.

Non-therapeutic Uses

Many studies have shown that expression and/or increased MMP activity correlate with tumor growth and neovascularization (Johnsen, M., et al, 1998 Curr. Opin. Cell Biol. 10:667-671; Hiraoka, N., 1998 Cell 91:439-442; Brown, P. D., 1993 J. Natl. Cancer Inst. 85:574-578). MMP inhibitors, both synthetic and endogenous, have been used to block angiogenic responses in vitro and in vivo (Moses, M. A., 1997 Stem Cells 15: 180-189; Murphy, A. N., 1993 J. Cell Physiol. 157:351-358).

In another aspect of the invention, it has been discovered that endothelial cell tube formation, and therefore angiogenesis, is correlated with the expression of matrix metalloproteinase MMP-9 expression in endothelial cells. Stimulation of endothelial cells with inducers, such as phorbol 12-myristate 13-acetate (PMA), result in a dose-dependent increase in endothelial cell tube formation as determined, for example, by measurement of average tube length in a HUVEC tube formation assay. Conversely, PPAR gamma ligands selectively inhibit expression and secretion of MMP-9 in endothelial cells. This inhibition is associated with a significant suppression of MMP-9 mRNA levels as analyzed by real-time RT-PCR. These discoveries form the basis of a method of assaying anti-endothelial cell tube forming properties or of assaying anti-angiogenesis properties of a PPARγ ligand test compound by determining the amount of endothelial cell tube formation in the presence of the test compound. For example, the amount of endothelial cell tube formation can be determined by comparing the amount of endothelial cell tube formation in the presence of the test compound to the amount of endothelial cell tube formation in the absence of the test compound, in for example a HUVEC tube assay format or against a standard curve of tube formation versus tube inhibitor concentration. Any suitable endothelial cell tube forming assay may be used, for example, the assay described herein or other known assay, such as described in Fisher, C., et al. 1994 Dev. Biol. 162:499-510; Schnaper, H. W., et al. 1993 J. Cell Physiol. 156:235-46; Ilan, N., et al, 1998 J. Cell Sci. 111:3621-31; and Davis, G. E., and Camarillo, C. W. 1996 Exp. Cell Res. 224:39-51. Alternatively, the anti-endothelial cell tube forming properties or anti-angiogenesis properties of a PPARγ ligand test compound can be determined by measuring the amount of MMP-9 mRNA in an endothelial cell sample cultured in the presence of the test compound. For example, the anti-endothelial cell tube forming properties or anti-angiogenesis properties of a PPARγ ligand test compound can be determined by comparing the amount of MMP-9 mRNA in an endothelial cell sample cultured in the absence of the test compound with the amount of MMP-9 mRNA in an endothelial cell sample cultured in the presence of the test compound or by comparing the test compound values to a standard curve prepared according to well known procedures. The mRNA may be determined by any known method, for example, using PCR or RT-PCR. The mRNA assay may be used as a primary screen for compounds which may have anti-angiogenic properties. For compounds which show activity in reducing MMP-9 mRNA, the primary screen may be followed by a secondary screen in which the active compounds are assayed in an endothelial cell tube forming assay to determine anti-tube forming activity. Compounds which show activity in either of these assays are potential candidates for anti-angiogenic therapeutic drugs since they have been shown to inhibit angiogenesis.

### EXAMPLES

Materials:

PGA₁, PGA₂, PGB₁, PGB₂, PGD₁, PGD₂, PGE₁, PGE₂, Troglitazone, Ciglitizone, WY 14643, LY 171883, and eicosatetraenoic acid (ETYA), 15-deoxy-delta^{12,14}-PGJ₂ (15d-PGJ₂) were from Cayman Chemical (Ann Arbor, Mich.). Human umbilical vein endothelial cells (HUVEC) were purchased from Clonetics (San Diego, Calif.) and maintained in Clonetics EGM media supplemented with 10% fetal bovine serum Type I rat tail collagen and recombinant basic fibroblast growth factor (bFGF) were purchased from Collaborative Research (Becton Dickinson labware, Bedford, Mass.). Recombinant VEGF was from Genentech (South San Francisco, Calif.). 10× medium 199 (M199) and PMA were purchased from Sigma Chemical Company. FBS was from Hyclone (Logan Utah). ITS (Insulin, transferrin and Selenium A), trypsin, and versene were from Gibco-BRL (Gaithersberg, Md.). Mouse monoclonal antisera to PPAR (E-8, SC-7273) were purchased from Santa Cruz Biotechnology (Santa Cruz, Calif.). Magnetic protein A beads (Dynabeads) were from Dynal (Lake Success, N.Y.).

### Example 1

### Endothelial Tube Assay

To determine which PPAR receptor was mediating the observed inhibition of tube formation, the effects of specific PPAR activators was assessed. Treatment of HUVEC with the specific PPAR gamma ligands, 15d-PGJ₂, troglitazone and ciglitizone dose-dependently inhibited tube formation with IC50 values of 2.8, 6.2, and 2.7 μM respectively. These IC50 values are in agreement with values reported for induction of adipocyte differentiation in vitro, the inhibition of macrophage and smooth muscle activation, and inhibition of breast cancer cell growth (Forman, B. et al, (1995) Cell 83:803-812; Kliewer, S. et al., (1995) Cell 83:813-819). In contrast, the PPAR alpha selective agonists, clofibrate, LY 171883, and WY 14643 at concentrations of up to 100 μM, and the PPAR beta selective activator, erucic acid (200 μM) did not significantly inhibit tube formation. These data confirm that ligands or agonists which cause PPAR gamma activation inhibit endothelial differentiation into tube-like structures in vitro, a model for angiogenesis.

Collagen gels were formed by mixing together ice-cold gelatin solution (10× M199, H₂O, 0.53M NaHCO₃, 200 mM L-glutamine, type I collagen, 0.1M NaOH, 100:27.2:50:10:750:62.5 by volume) and cells in 1× basal medium at a concentration of 3×106 cells/ml at a ratio of 4 volumes gelatin solution: 1 volume of cells. After gelation at 37 for 30 min, the gels were overlaid with IX basal medium consisting of M199 supplemented with 1%FBS, 1× ITS, 2 mM L-glutamine, 50 μg/ml ascorbic acid, 26.5 mM NaHCO₃, 100 U/ml penicillin and 100 U/mil streptomycin supplemented with 40 ng/ml BFGF, 40 ng/ml VEGF and 80 nM phorbol myristate acetate (PMA). All drugs were added to the IX basal medium immediately after gelation. To quantitate tube formation, the number of tubes per high power (20× field) was determined 48 hr after addition of the basal medium Five independent fields separated by 100 μm optical sections were assessed for each well. Cytotoxicity was assessed using an XTT kit from Boehringer Mannheim (Indianapolis, Ind.).

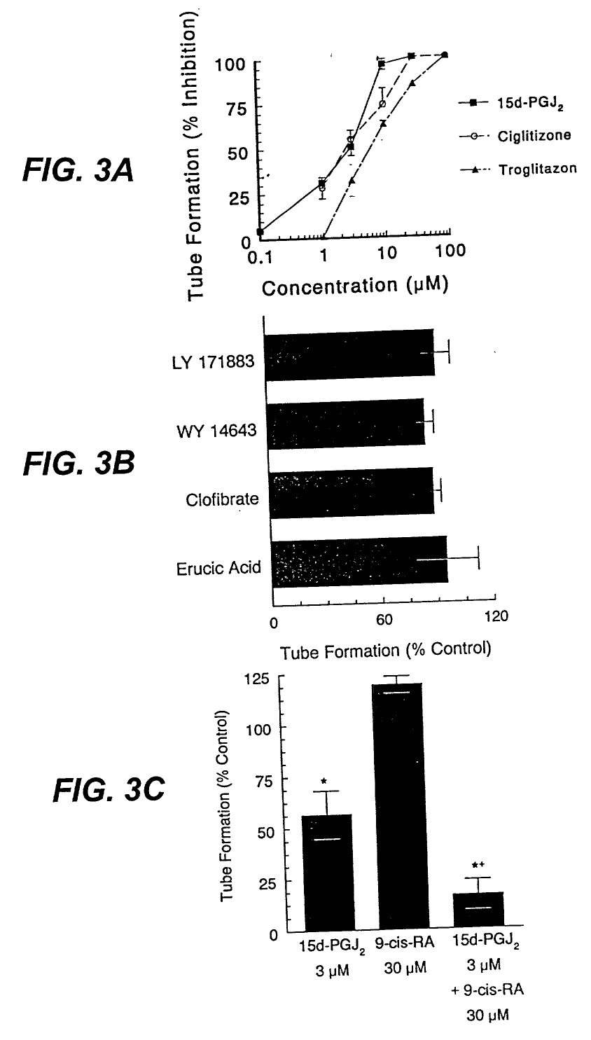

Synergistic inhibition of tube formation by HUVEC using 15d-PGJ₂ and 9-cis-retinoic acid was demonstrated as follows. 9-Cis-retinoic acid is known to be a selective ligand for the retinoic acid (RXR) receptor. Incubation of HUVEC with 9-cis-retinoic acid (30 μM) had no significant effect on tube formation. However, when tested in combination with 15d-PGJ₂ (3 μM), the inhibitory effects on tube formation were significantly greater than those observed with 15d-PGJ₂ (3 μM) alone (FIG. 3B). These observations are consistent with previous work demonstrating that PPAR gamma forms heterodimers with RXR receptors. PPAR-responsive elements (PPREs) composed of a directly repeating core-site separated by 1 nt, are recognized by the PPAR/RXR heterodimer.

### Example 2

### Reverse Transcription-Polymerase Chain Reaction (RT-PCR) Assays

Synthesis of complementary DNA (cDNA) was performed using 5 μg of total RNA extracted from human liver (positive control) or HUVEC cultured on collagen films by random primer according to the manufacturer's instructions (Stratagene Co., San Diego, Calif.). Subsequent amplifications of the partial cDNA encoding PPAR alpha, beta and gamma were performed using different amounts of reverse transcribed mixture (4 μl for liver PPAR alpha, beta and gamma and 5 μl for HUVEC PPAR alpha and beta and 6 μl for HUVEC PPAR gamma) as templates with specific oligonucleotide primers as follows: PPAR alpha sense 5′CCAGTATTTAGGACGCGGTCC3′ (SEQ ID NO: 1) and antisense 5′AAGTTCTTCAAGTAGGCCTGCG-3′ (SEQ ID NO: 2); PPAR beta sense 5′AACTGCAGATGGGCTGTGAC-3′ (SEQ ID NO: 3) and antisense 5′-GTCTCGATGTCGTGGATCAC3′ (SEQ ID NO: 4) and PPAR gamma sense 5′-TCTCTCCGTAATGGAAGACC-3′ (SEQ ID NO: 5) and antisense 5′GCATTATGAGACATCCCCAC-3′ (SEQ ID NO: 6). The expected sizes of PCR products for PPAR alpha, beta and gamma were 492 bp, 484 bp and 474 bp respectively. Negative controls for reverse transcription and PCR amplifications were included. The PCR mixtures were subjected to 35 cycles of amplification by denaturation (30 s at 94 C), hybridization (30 s at 55 C) and elongation (45 s at 55 C). The PCR products were analyzed by 1.5% agarose gel electrophoresis with ethidium bromide.

### Example 3

### Western Blot Assays

Confluent HUVEC were washed once with PBS, then scraped and pelleted by centrifugation. Pellets were lysed in ice-cold lysis buffer (20 mM Tris HCl, 150 mM NaCl, 2 mM EDTA, 1 mM 4-(2-aminoethyl)-benzenesulfonylfluoride, 40 U/ml aprotinin, 15 μg/ml leupeptin, 0.36 mM 1,10-phenanthroline, 1% Nonident NP-40 and 1% TritonX-100, pH 7.5). An aliquot equivalent to 100 μg protein was incubated 16 hr at 4 C with 20 ng of anti-PPAR gamma antisera in a total volume of 500 μl. Protein-A agarose beads (50 μl) were then added and the mixture incubated for 2 hr at 4 μC. The beads were washed 3 times with 500 μl of lysis buffer and the proteins remaining on the beads solubilized by heating at 100 C 5 min in 2× SDS sample buffer. The precipitated complexes were then analyzed by Western blotting.

### Example 4

### Corneal Angiogenesis Assay

A 1.5 mm incision was made approximately 1 mm from the center of the cornea of isoflurane-ketamine (60-80 mg/kg) xylazine (10-15 mg/kg) anesthetized Sprague-Dawley rats. Using a curved spatula, the incision was bluntly dissected through the stroma towards the outer canthus of the eye. A Hydron pellet (2×20 mm) containing VEGF (200 ng), sucralfate (100 μg) with or without (control) 15d-PGJ₂ (10 μg/pellet) was inserted into the base of the pocket. After surgery, the eyes were coated with gentamicin ointment. Animals are observed at 24-48 hr for the occurrence of nonspecific inflammation and then daily thereafter. At day 6, the animals were euthanized and injected with FITC-dextran to allow for visualization of the vasculature. Corneal whole mounts were made of the enucleated eyes and analyzed for neovascular area using the computer assisted image analysis.

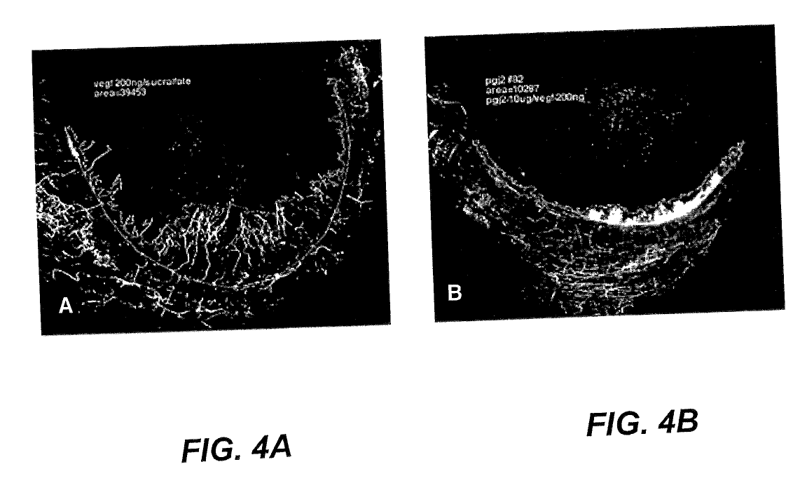

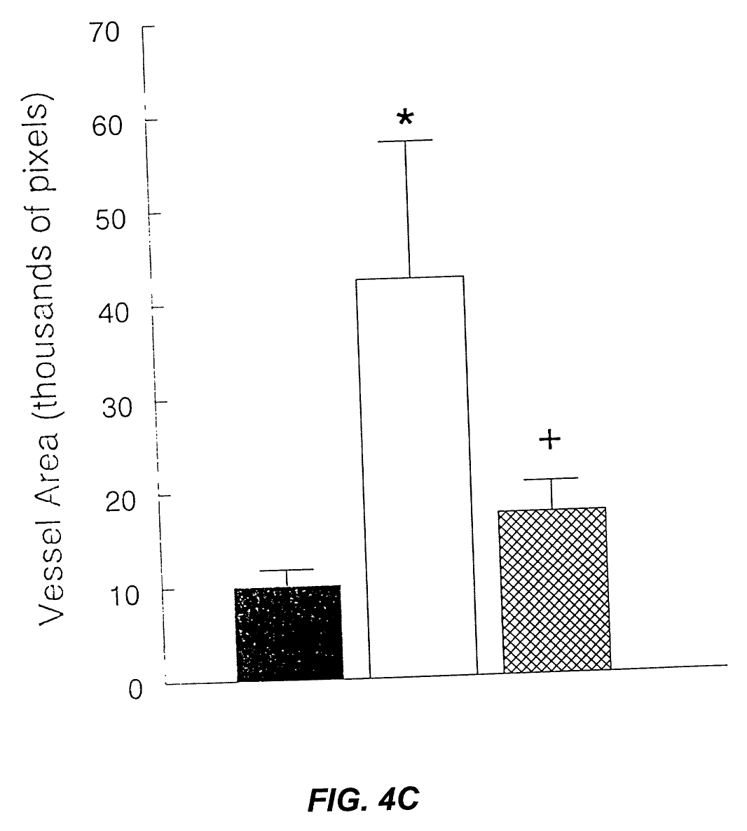

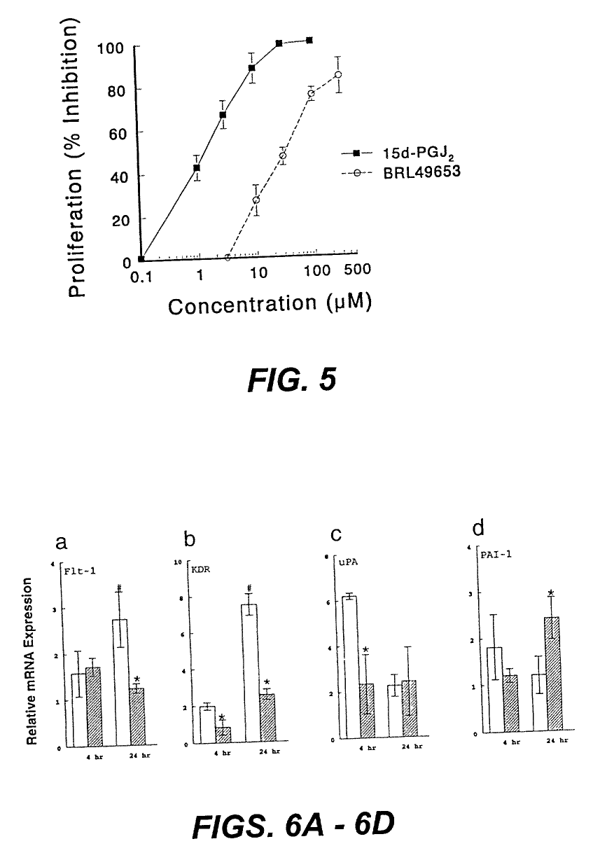

The in vivo anti-angiogenic effects of the prostanoid 15d-PGJ₂ were shown using Hydron pellets containing 200 ng recombinant VEGF, with or without 10 μg of 15d-PGJ₂, implanted into the corneas of Sprague-Dawley rats as described above. Summary data from this experiment show that pellets containing the combination 15d-PGJ₂ and VEGF produce a significant reduction in vessel length compared to the VEGF only (positive) controls (FIGS. 4A and 4B). Results from this experiment were repeated in a second independent experiment. These in vivo data are consistent with the in vitro results, i.e., activation of PPAR gamma produces strong inhibition of angiogenesis in endothelial tissue.

### Example 5

### Real Time RT-PCR (Taqman) Assay:

This technique has been used to quantitatively monitor mRNA expression and has been described in detail previously (Heid, C. A. et al., (1996) Genome Methods 6:986-994; Gerber, H. et al., (1997) J. Biol. Chem. 272:23659-23667). Total RNA was extracted from HUVEC cultured in three dimensional collagen gels or on collagen-coated flasks for various times in 1× basal medium consisting of M199 supplemented with 1%FBS, 1× ITS, 2 mM L-glutamine, 50 μg/ml ascorbic acid, 26.5 mM NaHCO₃, 100 U/ml penicillin and 100 U/ml streptomycin supplemented with 40 ng/ml bFGF (basic fibroblast growth factor), 40 ng/ml VEGF and 80 nM PMA (phorbol myristic acid). A gene-specific PCR oligonucleotide primer pair and an oligonucleotide probe labeled with a reporter fluorescent dye at the 5′-end and a quencher fluorescent dye at the 3′-end were designed using the Oligo 4.0 software (National Bioscience, Plymouth, Minn.) following guidelines suggested in the Taqman Model 7700 Sequence Detection instrument manual (PE Applied Biosystem). The primers and probes used were as follows: human PPARα gene forward primer 5′-GGACGTGCTTCCTGCTTCAT-3′ (SEQ ID NO: 7), reverse primer 5′-CACCATCGCGACCAGATG-3′ (SEQ ID NO: 8), and probe 5′-TTGGAGCTCGGCGCACAACCA-3′ (SEQ ID NO: 9); human PPARα gene forward primer 5′-TGACCTGCGGCAACTGG-3′ (SEQ ID NO: 10), reverse primer 5′-TTCGGTCTTCTTGATCCGCT-3′ (SEQ ID NO: 11), and probe 5′-CACCGAGCACGCCCAGATGATG-3′ (SEQ ID NO: 12); human PPARβ gene forward primer 5′-GCCAAGCTGCTCCAGAAAAT-3′ (SEQ ID NO: 13), reverse primer 5′-TGATCACCTGCAGTAGCTGCA-3′ (SEQ ID NO: 14), and probe 5′-ACAGACCTCAGACAGATTGTCACGGAACAC-3′ (SEQ ID NO: 15); human Flk/KDR gene forward primer 5′-CACCACTCAAACGCTGACATGTA-3′ (SEQ ID NO: 16), reverse primer 5′-CCAACTGCCAATACCAGTGGA-3′ (SEQ ID NO: 17), and probe 5′-TGCCATTCCTCCCCCGCATC-3′ (SEQ ID NO: 18); human Flt-1 gene forward primer 5′-ACCCAGATGAAGTTCCTTTGGA-3′ (SEQ ID NO: 19), reverse primer 5′-CCCAGTTTAGTCTCTCCCGG-3′ (SEQ ID NO: 20), and probe 5′-CCTTATGATGCCAGCAAGTGGGAGTTTG-3′ (SEQ ID NO: 21); human uPA gene forward primer 5′-ACGCTTGCTCACCAGAATGA-3′ (SEQ ID NO: 22), reverse primer 5′-GCGCACACCTGCCCTC-3′ (SEQ ID NO: 23), and probe 5′-ATTGCCTTGCTGAAGATCCGTTCCAA-3′ (SEQ ID NO: 24); human PAI-1 gene forward primer 5′-TCGTCCAGCGGGATCTGA-3′ (SEQ ID NO: 25), reverse primer 5′-GTGCTCCGGAACAGCCTG-3′ (SEQ ID NO: 26), and probe 5′-CCAGGGCTTCATGCCCCACTTCT-3′ (SEQ ID NO: 27); human glyceraldehyde-3-phosphate dehydrogenase (GAPDH) gene forward 5′-GAAGGTGAAGGTCGGAGTC-3′ (SEQ ID NO: 28), reverse 5′-GAAGATGGTGATGGGATTTC-3′ (SEQ ID NO: 29), and probe 5′-CAAGCTTCCCGTTCTCAGCC-3′ (SEQ ID NO: 30). Total RNA (100 ng) was added to a 50 μl RT-PCR reaction mixture according to the manufacturer's protocol (Roche Molecular Systems, Inc., Branchburg, N.J.). The thermal cycling conditions included 1 cycle at 48° C. for 30 min, 1 cycle at 95° C. for 10 min, 40 cycles at 95° C. for 15 s, at 60° C. for 1 min and finally hold at 25° C. for 2 min. Standard curves for expression of each gene were generated by serial dilution of the total RNA isolated from HUVEC. Expression of GAPDH gene was not significantly altered during the times (up to 48 hr) of incubation with drugs and vehicle. Therefore, the relative mRNA expression of each gene was normalized to the level of GAPDH in the same RNA preparation.

### Example 6

### Proliferation Assay:

HUVEC were seeded on collagen-coated 96-well plates at 6,000 cells/cm² in Clonetics EGM supplemented with 10% FBS, 2 mM L-glutamine, 100 U/ml penicillin and 100 U/ml streptomycin and allowed to attach for 4 hr. Medium was then replaced with 1× basal medium consisting of M199 supplemented with 1%FBS, 1× ITS, 2 mM L-glutamine, 50 μg/ml ascorbic acid, 26.5 nM NaHCO₃, 100 U/ml penicillin and 100 U/m streptomycin supplemented with 40 ng/ml bFGF, 40 ng/ml VEGF and 80 nM PMA. Cells were cultured in above medium in the presence of drugs or vehicle for 4 hr. 5 μl (100 μM) of 5′-bromo-2′-deoxyuridine (BrdU) was added in a final volume of 100 μl/well and cells were incubated for another 20 hr. BrdU incorporation was evaluated by an ELISA kit from Boehringer Mannheim (Indianapolis, Ind.).

Data are expressed as the mean±standard error. Statistic analysis was performed using one-way ANOVA (INSTAT, Graph Pad Software, Sorrento Valley, Calif.). Multiple comparisons against the control were analyzed using Bonferroni modification of Student's t-test to determine differences between groups. A p value <0.05 was accepted as significant.

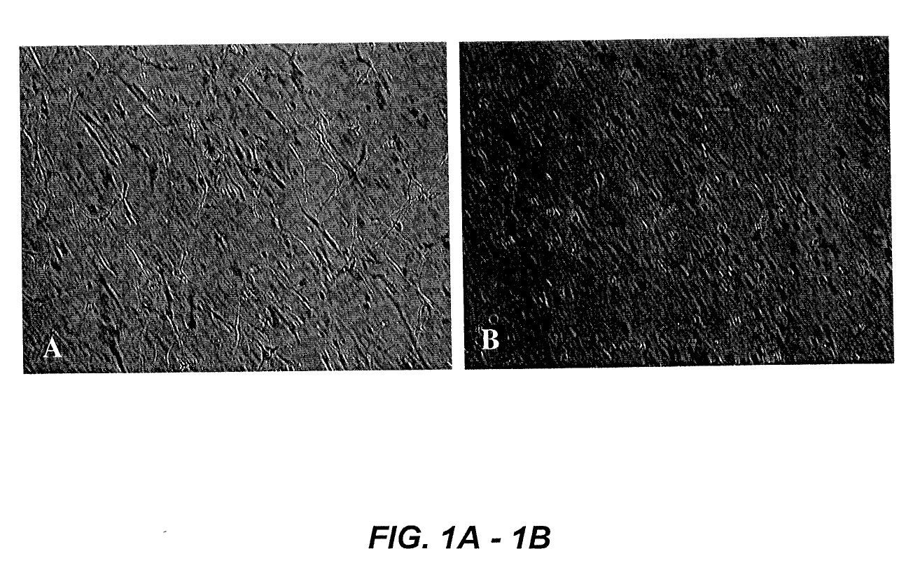

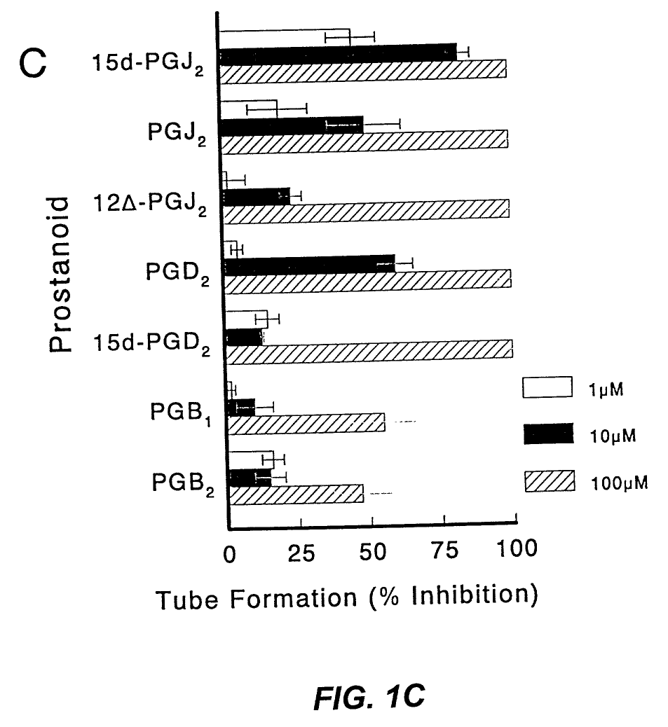

Examples 5 and 6 indicate that PPARγ ligands mediate the inhibitory effects of growth factors on endothelial cell proliferation. BrdU incorporation assays were performed to detect proliferation of endothelial cells cultured on type I collagen-coated surface in medium containing the growth factors, e.g. VEGF, bFGF and PMA, in the presence of vehicle or PPARγ ligands. As shown in FIG. 1, 15d-PGJ₂ and BRL49653 dose-dependently inhibited endothelial cell (HUVEC) proliferation with IC₅₀ of 2.4 and 15.7 μM, respectively. In contrast, PGE₁ and PGE₂ did not affect HUVEC proliferation. These results demonstrate that PPARγ ligands also repress or inhibit growth factor-induced endothelial cell proliferation. The present invention, therefore, is also directed to a method of inhibiting growth factor induced endothelial cell proliferation by contacting endothelial cells with an effective amount of a PPARγ ligand. Conditions associated with undesired vascularization and angiogenesis resulting from growth factor induced endothelial cell proliferation can be treated by administering the ligands in the manner described above.

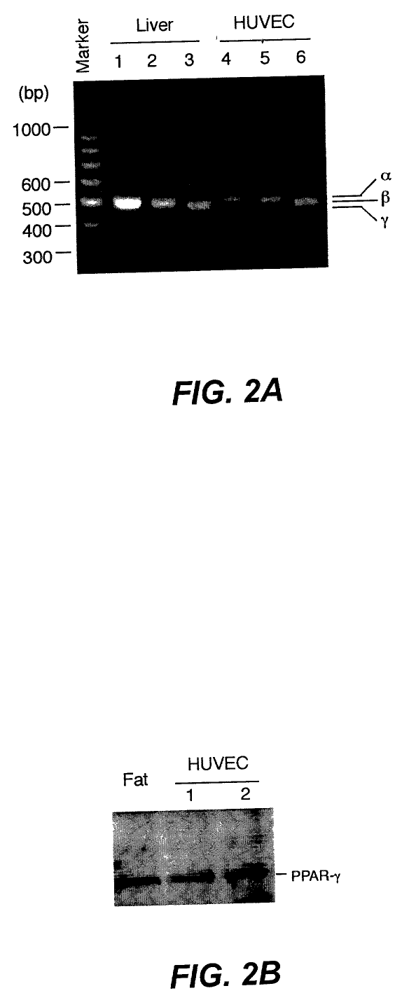

PPARγ ligands also regulate gene expression events associated with angiogenesis. Flk/KDR and Flt-1 are two structurally related endothelial cell tyrosine kinase receptors for VEGF. The importance of these two receptors during angiogenesis has been clearly demonstrated by the findings that KDR functions as a transducer to signal endothelial cell proliferation and differentiation and that Flt-1 is a critical survival factor involved in endothelial cell morphogenesis (Fong, G. H. et al., (1995) Nature (London) 376:66-70; Ferrara, N. et al., (1997) Endocr. Rev. 18:4-25; Ilan, N. et al., (1998) J. Cell Sci. 111:3621-3631). We examined whether activation of PPARγ with a ligand therefor alters Flt-1 and KDR gene expression using real time quantitative RT-PCR. Both KDR and Flt-1 mRNA were up-regulated by the mixture of growth factors in HUVEC grown in three dimensional collagen gels (FIGS. 2a and 2b, open bars). 15d-PGJ₂ inhibited the induction of both VEGF receptor mRNA in HUVEC cultured in three dimensional collagen gels (FIGS. 2a and 2b, hatched bars). Similar data were obtained when HUVEC were grown on type I collagen-coated surface with growth factor stimulation. It is also well known that the production of proteases (e.g. plasminogen activators) and their inhibitors (e.g. plasminogen activator inhibitor I, PAI-1) is correlated with endothelial cell degradation of extracellular matrix and migration, the two critical steps of the angiogenic processes (34). We analyzed the effects of 15d-PGJ₂ on gene expression of urokinase type plasminogen (uPA) and PAI-1 in three dimensional collagen gels. Treatment of HUVEC with 15d-PGJ₂ reduced uPA mRNA at 4 hr and increased PAI-1 gene expression at 24 hr, respectively (FIGS. 2c and 2d, hatched bars). Taken together, these observations indicate molecular mechanisms by which PPARγ ligands mediate inhibition of angiogenesis.

### Example 7

### MMP-9 Correlation with Angiogenesis

Endothelial Tube Assay:

Collagen gels were formed by mixing together ice-cold gelation solution (IOX M199, H₂O, 0.53M NaHCO₃, 200 mM L-glutamine, type I collagen, 0.1M NaOH, 100:27.2:50:10:750:62.5 by volume) and cells in 1× basal medium (see below) at a concentration of 3×10⁶ cells/ml at a ratio of 4 volumes gelation solution: 1 volume of cells (18). After gelation at 37° C. for 30 min, the gels were overlaid with 1× basal medium consisting of M199 supplemented with 11%FBS, 1× ITS, 2 mM L-glutamine, 50 mg/ml ascorbic acid, 26.5 mM NaHCO₃, 100 U/ml penicillin and 100 U/ml streptomycin supplemented with 40 ng/ml bFGF, 40 ng/ml VEGF, 80 nM PMA or the combination of PMA (80 nM), bFGF and VEGF (40 ng/ml each). All drugs were added to the 1× basal medium immediately after gelation. To quantify tube formation, the average tube length was determined by measuring total tube length (long axis) in three consecutive layers of a low power (10×) field using Openlab software (Improvision Inc, England). Each layer was separated by 100 μm.

Real Time RT-PCR (Taqman) Assay:

Total RNA was extracted from HUVEC cultured in 3-D type I collagen gels for various times in 1× basal medium supplemented with 40 ng/ml bFGF, 40 ng/ml VEGF, 80 mM PMA or the combination of PMA (80 nM), bFGF and VEGF (40 ng/mil each). A gene-specific PCR oligonucleotide primer pair and an oligonucleotide probe labeled with a reporter fluorescent dye at the 5′-end and a quencher fluorescent dye at the 3′-end were designed using the Oligo 4.0 software (National Bioscience, Plymouth, Minn.) following guidelines suggested in the Taqman Model 7700 Sequence Detection instrument manual (PE Applied Biosystem). For all mRNAs evaluated a sample of total RNA (100 ng) was added to a 50 μl RT-PCR reaction mixture according to the manufacturer's protocol (Roche Molecular Systems, Inc., Branchburg, N.J.). The thermal cycling conditions included 1 cycle at 48° C. for 30 min, 1 cycle at 95° C. for 10 min, 40 cycles at 95° C. for 15 s, at 60° C. for 1 min, and finally hold at 25° C. for 2 min. Standard curves for expression of each gene were generated by serial dilution of the total RNA isolated from U937 cells. Expression of GAPDH gene was not significantly altered during the times (up to 48 h) of incubation with drugs and vehicle. Therefore, the relative mRNA expression of each gene was normalized to the level of GAPDH in the same RNA preparation.

Zymographic Assay:

The presence of gelatinases MMP-2 and -9 in conditioned media from cultured HUVEC in 3-D type I collagen gels was analyzed by zymography. Media from different treatments or from different time points were concentrated and then 10 μl aliquots were mixed with 2 × sample buffer without reducing agent or heating. Samples were loaded on a 10% polyacrilamide gel containing 0.1% gelatin (Novex, San Diego, Calif.) for electrophoresis. In addition to using regular molecular weight markers the MMP-2 and -9 zymographic standards (Chemicon, Temecula, Calif.) were used as standards for gelatinases. The gels were incubated for 30 min at room temperature in renaturing buffer and in developing buffer for overnight at 37° C., and then stained with 0.25% Coomassie brilliant blue (Sigma). Gelatinase activity was identified as lightly stained or clear bands following destaining.

Western Blot Assays: Aiquots of 20 μl of concentrated media were denatured with SDS under reducing conditions and applied to 10% polyacrylamide gels. After separation by electrophoresis, the proteins were electroblotted onto Optitran nitrocellulose transfer immobilization membrane (Schleicher & Schuell, Keene N.H.). MMP-1 proteins were revealed by a specific monoclonal antibody against human MMP-1 (MAB3307) (Chemicon, Temecula, Calif.) using ECL chemiluminescence kit (Amershar, Clearbrook Ill.).

Statistics:

Data are expressed as the mean±S.E.M. Statistical analysis was performed using one-way analysis of variance (INSTAT, GraphPad Software, Sorrento, Calif.). Multiple comparisons against the control were analyzed using Bonferroni modification of Student's t test to determine differences between groups. A p value <0.05 was accepted as significant.

HUVEC morphogenesis expression correlates with MMP mRNA expression by bFGF, VEGF and PMA in 3-D type I collagen gels. HUVEC, when cultured in 3-D type I collagen gels in the presence of the combination of PMA, bFGF and VEGF, differentiate into tube-like structures with frequent lumens. When HUVEC were suspended in 3-D type collagen gels in the presence of PMA (80 nM), a sequential series of morphogenic alterations were observed. By 4 h, large vacuoles could be seen in the majority of cells. Frequent small finger-like projections were observed. At 24 h, many of cells exhibited an elongated morphology, and short tube-like structures were seen and some of the cells had connected with each other. When HUVEC treated with combination of PMA (80 nM), bFGF (40 ng/ml) and VEGF (40 ng/ml) for 24 h, longer tube-like structures were observed and many of the structures were inter-connected.

To determine the correlation of MMP mRNA expression with HUVEC morphogenesis, total RNA was extracted from HUVEC cultured in 3-D collagen gels for 4 h or 24 h treated with bFGF (40 ng/ml), VEGF (40 ng/ml), PMA (80 nM) or PMA (80 nM) in combination with bFGF and VEGF (40 ng/ml each) and were analyzed by real-time quantitative RT-PCR (Taqman). MMP-9 mRNA levels were at or below the limits of detection in the bFGF and VEGF-stimulated groups at 4 h and 24 h. However, HUVEC treated with PMA or PMA in combination with bFGF and VEGF demonstrated detectable MMP-9 mRNA levels at 4 h and an increase of more than 2-fold between 24 h and 4 h in the PMA group, and nearly 4-fold in the combination of PMA, bFGF and VEGF treated group. In a second series of experiments, a more detailed time course (4, 8, 16, 24 and 48 h) of the MMP mRNA expressions was examined. MMP-9 mRNA reached maximal levels at 24 h and then declined slightly at 48 h. Levels of MMP-3 mRNA level were much lower than that of MMP-2 and decreased slightly between 24 h versus 4 h in all of the treatment groups. There were no significant differences in MMP-3 expression between any of the groups.

The content of MMP-2 and -9 in culture supernatants was assessed by gelatin zymography. Both the latent (72 kD) and active forms (66 and 62 kD bands) of MMP-2 accumulated in a time-dependent manner in culture supernatant from bFGF, VEGF, PMA or PMA in the combination with bFGF and VEGF treated HUVEC. Supernatants from cells treated with PMA, in the absence or presence of FGF and VEGF, contained more active MMP-2 than supernatants from cells incubated with either growth factor alone. However, the zymogen form of MMP-9 (92 kD) was detected only in supernatants from HUVEC treated with PMA or PMA in combination with bFGF and VEGF. There was a time-dependent increase of accumulation of MMP-1, as assessed by Western blot analysis in supernatants collected from HUVEC cultured in 3-D collagen gels in the presence of bFGF, VEGF, PMA or PMA in combination with bFGF and VEGF.

PMA dose-dependently induced HUVEC tube formation and MMP-2 and -9 secretion. Stimulation of HUVEC in 3-D gels with different concentrations of PMA (0.8, 8, 80 and 800 nM) for 24 h resulted in a dose-dependent increase in tube formation as determined by measurement of the average tube length. Maximal induction of tube formation was observed with 80 nM PMA. This PMA-induced HUVEC morphogenesis directly correlated with a dose-dependent stimulation of MMP-2 and -9 secretion into the culture medium.

PPARγ ligands selectively inhibited expression and secretion of MMP-2 and -9. PPARγ ligands inhibit HUVEC tube formation in vitro as well as VEGF-stimulated angiogenesis in vivo as described above. To investigate the effect of PPARγ activation on MMP mRNA expression and protein secretion, HUVEC were cultured in 3-D collagen gels supplemented with medium containing PMA (80 nM) or PMA (80 nM) in combination with bFGF and VEGF(40 ng/ml each) in the presence of vehicle or selective ligands for PPARγ (15d-PGJ₂, ciglitizone) or PPARα (WY 14643, clofibrate). After 24 h incubation, culture supernatants were collected and RNA from treated cells were isolated. The PPARγ selective ligands, 15d-PGJ_{□} (10 μM) or ciglitizone (10 μM), blocked PMA or PMA in combination with bFGF and VEGF induced HUVEC tube formation in 3-D collagen gels. This inhibition was associated with a significant suppression of MMP-2 and -9 mRNA levels as analyzed by real-time RT-PCR. Analysis of protein secretion by gelatin zymography showed a similar inhibition of HUVEC secretion of MMP-2 (latent and active forms) and MMP-9 zymogen by both PPARγ ligands. Further experiments showed that PPARγ ligands dose-dependently blocked PMA stimulated MMP-2 and -9 protein secretion by HUVEC cultured in 3-D gels. In contrast, the PPARα ligands, WY 14643 (100 μM) and clofibrate (100 μM), which do not inhibit HUVEC tube formation, did not alter the mRNA expression or protein secretion of MMP-2 and -9. Neither PPARγ nor PPARα ligands inhibited the mRNA levels or the protein secretion of MMP-1. 15d-PGJ₂ treatment had no effect on MMP-3 mRNA expression in the PMA-treated group and increased MMP-3 mRNA levels in HUVEC cultured with combination of bFGF, VEGF and PMA at 24 h.

PPARγ ligands did not affect TIMP-1 and -2 mRNA expression. To examine whether PPARγ ligands also altered the proteolytic balance of MMPs by inducing the expression of tissue inhibitors of metalloproteinases (TIMPs), we analyzed TIMP-1 and -2 mRNA expression in HUVEC treated with the combination of bFGF, VEGF and PMA and the PPARγ ligand15d-PGJ₂ (10 μM). TIMP-1 mRNA expression increased nearly 3 fold at 24 h compared to 4 h. Treatment of HUVEC with 15d-PGJ₂ did not increase the expression of TIMP-1 at 4 h or 24 h. TIMP-2 mRNA levels were reduced in the 15d-PGJ₂ treated group compared to the vehicle treated group at 4 h.

MMPs play important roles in the degradation of basement membrane and proteolytic remodeling of matrix proteins involved in angiogenesis. This example shows that induction of MMP-9 is an important component for HUVEC differentiation into tube-like structures in 3-D type I collagen gels, even though both MMP-1 and -2 are simultaneously upregulated. PPARγ ligands, which inhibit angiogenesis in vitro and in vivo, selectively suppress the mRNA expression and enzyme secretion of both MMP-2 and -9 without a concomitant inhibition of MMP-1 and -3 expression or secretion and induction of TIMP-1 and -2 mRNA levels.

Induction of MMP expression and/or secretion has been previously implicated in in vitro angiogenesis (Fisher, C., et al. 1994, Dev. Biol. 162:499-510). This experiment shows that MMP-9 is an important component for HUVEC tube formation. MMP-9 has recently been found to play important role in regulation of angiogenesis. Direct evidence from an in vitro study showed a specific and time- and dose-dependent induction of MMP-9 in bovine aortic endothelial cells cultured in collagen gels by thrombospondin-1, a modulator of angiogenesis (Qian, X., et al., 1997 Exp. Cell Res. 235:403-12). A null mutation in MMP-9 results in an abnormal pattern of skeletal growth plate vascularization and ossification in the MMP-9 null mice (Vu, T. H., et al., 1998 Cell 93:411-22). In contrast, MMP-2 null animals appear to be both developmentally and physiologically normal (Itoh, T., et al. 1997 J. Biol. Chem. 272:22389-92). MMP-9 is highly expressed in a number of angiogenesis-related pathological states including rheumatoid arthritis, tumor metastasis and comeal ulcers, providing further support for a role of MMP-9 in angiogenesis (Vu, T. H., and Werb, Z. 1998. Gelatinase B: structure, regulation and function. In Matrix Metalloproteinases (ed. W. C. Parks and R. P. Mecham), pp. 115-148. San Diego: Academic Press).

Without being bound by a particular theory, the inhibition of the induced expression and secretion of MMP-2 and -9 by PPARγ ligands (e.g., 15d-PGJ₂ and ciglitizone) provide mechanistic insights into the anti-angiogenic effect of these drugs and further support the concept that gelatinases play major role in morphogenesis of endothelial cells into tube-like structures. Direct inhibition of MMP, in particular MMP-9, mRNA expression by PPARγ ligands appears to be the major pathway for suppression of MMP expression and proteolytic activity, contributing to inhibition of HUVEC tube formation.

Obviously, numerous modifications and variations of the present invention are possible in light of the above teachings. It is therefore to be understood that within the scope of the appended claims, the invention may be practiced otherwise than as specifically described herein.

What is claimed:

1. A method of inhibiting angiogenesis comprising:
(a) identifying a patient in need of an angiogenesis inhibitor; and
(b) administering to the patient a therapeutically effective amount of a PPAR gamma ligand, wherein angiogenesis is inhibited in the patient.

2. The method of claim 1, wherein the patient is a mammal.

3. The method of claim 2, wherein the mammal is human.

4. The method of claim 1, wherein the therapeutically effective amount of a PPAR gamma ligand is an angiogenesis inhibiting amount.

5. The method of claim 1, further comprising administering a therapeutically effective amount of an RXR receptor ligand.

6. The method of claim 1, wherein the PPAR gamma ligand is selected from the group consisting of (+)-5-[[4-[(3,4-dihydro-6-hydroxy-2,5,7,8-tetramethyl-2H-1-benzopyran-2-yl)methoxy]phenyl]methyl]-2,4-thiazolidinedione: (troglitazone); 5-[4-[2-(5-ethylpyridin-2-yl)ethoxyl]benzyl]thiadiazolidine-2,4-dione: (pioglitazone); 5-[4-[(1-methylcyclohexyl)methoxy]benzyl]thiadiazolidine-2,4-dione: (ciglitazone); 4-(2-naphthylmethyl)-1,2,3,5-oxathiadiazole-2-oxide; 5-[4-[2-[N-(benzoxazol-2-yl)-N-methylamino]ethoxy]benzyl]-5-methylthiazolidine-2,4-dione; 5-[4-[2-[2,4-dioxo-5-phenylthiazolidin-3-yl) ethoxy]benzyl]thiazolidine-2,4-dione; 5-[4-[2[N-methyl-N-(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chlorophenyl) ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiadiazolidine-2,4-dione; 5-[4-[2-(5-methyl-2-phenyloxazol-4-yl)ethoxyl]benzyl]thiadiazolidione-2,4-dione; 5-[(2-benzyl-2,3-dihydrobenzopyran)-5-ylmethyl]thiadiazoline-2,4-dione: (englitazone); 5-[[2-(2-naphthylmethyl)benzoxazol]-5-ylmethyl]thiadiazoline-2,4-dione; 5-[4-[2-(3-phenylureido)ethoxyl]benzyl]thiadiazoline-2,4-dione; 5-[4-[2-[N-(benzoxazol-2-yl)-N-methylamino]ethoxy]benzy]thiadiazoline-2,4-dione; 5-[4-[3-(5-methyl-2-phenyloxazol-4-yl)propionyl]benzyl]thiadiazoline-2,4-dione; 5-[2-(5-methyl-2-phenyloxazol-4-ylmethyl)benzofuran-5-ylmethyl]-oxazolidine-, 4-dione; 5-[4-[2-[N-methyl-N-(2-pyridyl)amino]ethoxy]benzyl]thiazolidine-2,4-dione (BRL 49653); and 5-[4-[2-[N-(benzoxazol-2-yl)-N-methylamino]ethoxy]benzyl]-oxazolidine-2,4-dione.

7. The method of claim 1, wherein the PPAR gamma ligand is selected from the group consisting of PGA₁, PGA₂, PGB₁, PGB₂, PGD₁, PGD₂, PDJ₂, 15-deoxy-12,14-delta-PGJ₂, and 12-delta-PGJ₂.

8. The method of claim 1, wherein the PPAR gamma ligand is a fatty acid containing about 10 to about 26 carbon atoms and zero to about 6 carbon-carbon double bonds or carbon-carbon triple bonds.

9. The method of claim 1, wherein the patient has a disease or disorder characterized by undesirable excessive neovascularization.

10. The method of claim 9, wherein the disease or disorder is selected from the group consisting of a neoplasm, rheumatoid arthritis, psoriasis, atherosclerosis, diabetic and other retinopathy, endometriosis, retrolental fibroplasia, age-related macular degeneration, neovascular glaucoma, thyroid hyperplasia, tissue transplantation, lung inflammation, obesity, and chronic inflammation.

11. The method of claim 10, wherein the neoplasm is a solid malignant tumor.

12. A method of inhibiting angiogenesis in a patient, comprising:
(a) identifying a patient with a disease or disorder selected from the group consisting of a neoplasm, rheumatoid arthritis, psoriasis, atherosclerosis, thyroid hyperplasia, endometriosis, lung inflammation, obesity, and chronic inflammation; and
(b) administering an angiogenesis inhibiting amount of a PPAR gamma ligand, wherein angiogenesis is inhibited in the patient.

13. The method of claim 12, wherein the patient is a mammal.

14. The method of claim 13, wherein the mammal is a human.

15. The method of claim 12, further comprising administering a therapeutically effective amount of an RXR receptor ligand.

16. The method of claim 12, wherein the PPAR gamma ligand is selected from the group consisting of (+)-5-[[4-[(3,4-dihydro-6-hydroxy-2,5,7,8-tetramethyl-2H-1-benzopyran-2-yl)methoxy]phenyl]methyl]-2,4-thiazolidinedione: (troglitazone); 5-[4-[2-(5-ethylpyridin-2-yl)ethoxyl]benzyl]thiadiazolidine-2,4-dione: (pioglitazone); 5-[4-[(1-methylcyclohexyl)methoxy]benzyl]thiadiazolidine-2,4-dione: (ciglitazone); 4-(2-naphthylmethyl)-1,2,3,5-oxathiadiazole-2-oxide; 5-[4-[2-[N-(benzoxazol-2-yl)-N-methylamino]ethoxy]benzyl]-5-methylthiazolidine-2,4-dione; 5-[4-[2-[2,4-dioxo-5-phenylthiazolidin-3-yl) ethoxy]benzyl]thiazolidine-2,4-dione; 5-[4-[2[N-methyl-N-(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chlorophenyl) ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiadiazolidine-2,4-dione; 5-[4-[2-(5-methyl-2-phenyloxazol-4-yl)ethoxyl]benzyl]thiadiazolidione-2,4-dione; 5-[(2-benzyl-2,3-dihydrobenzopyran)-5-ylmethyl]thiadiazoline-2,4-dione: (englitazone); 5-[[2-(2-naphthylmethyl)benzoxazol]-5-ylmethyl]thiadiazoline-2,4-dione; 5-[4-[2-(3-phenylureido)ethoxyl]benzyl]thiadiazoline-2,4-dione; 5-[4-[2-[N-(benzoxazol-2-yl)-N-methylamino]ethoxy]benzy]thiadiazoline-2,4-dione; 5-[4-[3-(5-methyl-2-phenyloxazol-4-yl)propionyl]benzyl]thiadiazoline-2,4-dione; 5-[2-(5-methyl-2-phenyloxazol-4-ylmethyl)benzofuran-5-ylmethyl]-oxazolidine-, 4-dione; 5-[4-[2-[N-methyl-N-(2-pyridyl)amino]ethoxy]benzyl]thiazolidine-2,4-dione (BRL 49653); and 5-[4-[2-[N-(benzoxazol-2-yl)-N-methylamino]ethoxy]benzyl]-oxazolidine-2,4-dione.

17. The method of claim 12, wherein the PPAR gamma ligand is selected from the group consisting of PGA₁, PGA₂, PGB₁, PGB₂, PGD₁, PGD₂, PDJ₂, 15-deoxy-12,14-delta-PGJ₂, and 12-delta-PGJ₂.

18. The method of claim 12, wherein the PPAR gamma ligand is a fatty acid containing about 10 to about 26 carbon atoms and zero to about 6 carbon-carbon double bonds or carbon-carbon triple bonds.

19. A method of providing an article of manufacture for inhibiting angiogenesis in a patient, comprising the step of providing:
(a) a container comprising a composition having a therapeutically effective amount of a PPAR gamma ligand therein; and
(b) an indication that the composition can be used to inhibit angiogenesis.

20. The method of claim 19, wherein the patient is a mammal.

21. The method of claim 20, wherein the mammal is human.

22. The method of claim 19, wherein the therapeutically effective amount of a PPAR gamma ligand is an angiogenesis inhibiting amount.

23. The method of claim 19, further comprising the step of providing a composition comprising a therapeutically effective amount of an RXR receptor ligand.

24. The method of claim 19, wherein the PPAR gamma ligand is selected from the group consisting of (+)-5-[[4-[(3,4-dihydro-6-hydroxy-2,5,7,8-tetramethyl-2H-1-benzopyran-2-yl)methoxy]phenyl]methyl]-2,4-thiazolidinedione: (troglitazone); 5-[4-[2-(5-ethylpyridin-2-yl)ethoxyl]benzyl]thiadiazolidine-2,4-dione: (pioglitazone); 5-[4-[(1-methylcyclohexyl)methoxy]benzyl]thiadiazolidine-2,4-dione: (ciglitazone); 4-(2-naphthylmethyl)-1,2,3,5-oxathiadiazole-2-oxide; 5-[4-[2-[N-(benzoxazol-2-yl)-N-methylamino]ethoxy]benzyl]-5-methylthiazolidine-2,4-dione; 5-[4-[-[2,4-dioxo-5-phenylthiazolidin-3-yl) ethoxy]benzyl]thiazolidine-2,4-dione; 5-[4-[2[N-methyl-N-(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chlorophenyl) ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiadiazolidine-2,4-dione; 5-[4-[2-(5-methyl-2-phenyloxazol-4-yl)ethoxyl]benzyl]thiadiazolidione-2,4-dione; 5-[(2-benzyl-2,3-dihydrobenzopyran)-5-ylmethyl]thiadiazoline-2,4-dione: (englitazone); 5-[[2-(2-naphthylmethyl)benzoxazol]-5-ylmethyl]thiadiazoline-2,4-dione; 5-[4-[2-(3-phenylureido)ethoxyl]benzyl]thiadiazoline-2,4-dione; 5-[4-[2-[N-(benzoxazol-2-yl)-N-methylamino]ethoxy]benzy]thiadiazoline-2,4-dione; 5-[4-[3-(5-methyl-2-phenyloxazol-4-yl)propionyl]benzyl]thiadiazoline-2,4-dione; 5-[2-(5-methyl-2-phenyloxazol-4-ylmethyl)benzofuran-5-ylmethyl]-oxazolidine-, 4-dione; 5-[4-[2-[N-methyl-N-(2-pyridyl)amino]ethoxy]benzyl]thiazolidine-2,4-dione (BRL 49653); and 5-[4-[2-[N-(benzoxazol-2-yl)-N-methylamino]ethoxy]benzyl]-oxazolidine-2,4-dione.

25. The method of claim 19, wherein the PPAR gamma ligand is selected from the group consisting of PGA₁, PGA₂, PGB₁, PGB₂, PGD₁, PGD₂, PDJ₂, 15-deoxy-12,14-delta-PGJ₂, and 12-delta-PGJ₂.

26. The method of claim 19, wherein the PPAR gamma ligand is a fatty acid containing about 10 to about 26 carbon atoms and zero to about 6 carbon-carbon double bonds or carbon-carbon triple bonds.

27. The method of claim 19, wherein a label provides the indication.

28. An article of manufacture for inhibiting angiogenesis prepared by the method of claim 19.

29. An article of manufacture for inhibiting angiogenesis comprising:
(a) a container;
(b) a composition within the container comprising a therapeutically effective amount of a PPAR gamma ligand; and
(c) an indication that the composition can be used to inhibit angiogenesis.
